SEQUENCE LISTING (15 sequences)

SEQ ID NO: 1 - protein, 23 aa, synthetic construct
  REGION 1..23: note = NB-2Ni peptide
  VAR_SEQ 1..5: note = X is absent or any amino acid of the L- or D- configuration
  VAR_SEQ 7: note = X is any amino acid of the L- or D-configuration
  VAR_SEQ 9: note = X is any amino acid of the L- or D-configuration
  VAR_SEQ 11: note = X is any amino acid of the L- or D-configuration
  VAR_SEQ 12..16: note = X is absent or any amino acid of the L- or D- configuration
  VAR_SEQ 18: note = X is any amino acid of the L- or D-configuration
  VAR_SEQ 19..23: note = X is absent or any amino acid of the L- or D-configuration
XXXXXCXCXCXXXXXXCXXXXXX

SEQ ID NO: 2 - protein, 13 aa, synthetic construct
  REGION 1..13: note = NB-2Ni peptide
CNCGCGNNNDRCG

SEQ ID NO: 3 - protein, 13 aa, synthetic construct
  REGION 1..13: note = NB-2Ni peptide
CNCGCGNWNDRCG

SEQ ID NO: 4 - protein, 13 aa, synthetic construct
  REGION 1..13: note = NB-2Ni peptide
CNCACGNWNDRCG

SEQ ID NO: 5 - protein, 14 aa, synthetic construct
  REGION 1..14: note = NB-2Ni peptide
GCNCGCGNNNDRCG

SEQ ID NO: 6 - protein, 15 aa, synthetic construct
  REGION 1..15: note = NB-2Ni peptide
CNCGCGNNNNNDRCG

SEQ ID NO: 7 - protein, 16 aa, synthetic construct
  REGION 1..16: note = NB-2Ni peptide
GGCNCGCGNWNDRCGG

SEQ ID NO: 8 - protein, 13 aa, synthetic construct
  REGION 1..13: note = NB-2Ni peptide
  VAR_SEQ 7..9: note = amino acids in the D-configuration
CNCGCGNNNDRCG

SEQ ID NO: 9 - protein, 13 aa, synthetic construct
  REGION 1..13: note = NB-2Ni peptide
CGCGCGWHGGRCG

SEQ ID NO: 10 - protein, 8 aa, synthetic construct
  REGION 1..8: note = NB-2Ni peptide
  VAR_SEQ 5..7: note = amino acid of the D-configuration
CRCWCHCG

SEQ ID NO: 11 - protein, 8 aa, synthetic construct
  REGION 1..8: note = NB-2Ni peptide
  VAR_SEQ 5..7: note = amino acids of the D-configuration
CHCWCHCG

SEQ ID NO: 12 - protein, 14 aa, synthetic construct
  REGION 1..14: note = NB-2Ni peptide
  VAR_SEQ 2: note = X is any amino acid of the L- or D- configuration
  VAR_SEQ 4: note = X is any amino acid of the L- or D- configuration
  VAR_SEQ 6..7: note = X is any amino acid of the L- or D- configuration
  VAR_SEQ 8..11: note = X is absent or any amino acid of the L- or D- configuration
  VAR_SEQ 14: note = X is any amino acid of the L- or D- configuration
CXCXCXXXXXXRCX

SEQ ID NO: 13 - protein, 13 aa, synthetic construct
SNCGCGNNNDRSG

SEQ ID NO: 14 - protein, 15 aa, synthetic construct
CNCGCGNWNDRCGGK

SEQ ID NO: 15 - protein, 16 aa, synthetic construct
CNCGCGNWNDRCGGGK

(19) United States
(12) Patent Application Publication
(10) Pub. No.: US 2023/0302437 A1
(43) Pub. Date: Sep. 28, 2023

(54) A MINIMAL CATALYTIC DI-NICKEL PEPTIDE CAPABLE OF SUSTAINED HYDROGEN EVOLUTION AND METHODS OF USE THEREOF

(71) Applicant: RUTGERS, THE STATE UNIVERSITY OF NEW JERSEY, New Brunswick, NJ (US)

(72) Inventors: Jennifer Timm, North Brunswick, NJ (US); Paul G. Falkowski, Princeton, NJ (US); Vikas Nanda, Highland Park, NJ (US); Alexei M. Tyryshkin, Yardley, PA (US); Saroj Poudel, New Brunswick, NJ (US); Joshua A. Mancini, North Royalton, OH (US); Douglas H. Pike, New Brunswick, NJ (US)

(73) Assignee: RUTGERS, THE STATE UNIVERSITY OF NEW JERSEY, New Brunswick, NJ (US)

(21) Appl. No.: 18/047,822

(22) Filed: Oct. 19, 2022

Related U.S. Application Data
(60) Provisional application No. 63/257,464, filed on Oct. 19, 2021.

(51) Int. Cl. B01J 31/18; B01J 35/00; C01B 3/22; C07K 7/08; C12P 3/00; C12M 1/00; C12M 1/107

(52) U.S. Cl. CPC B01J 31/1805; B01J 35/004; C01B 3/22; C07K 7/08; C12P 3/00; C12M 21/02; C12M 29/04; C12M 21/04; B01J 2531/0208; B01J 2531/847; B01J 2231/005; C01B 2203/0277; C01B 2203/1058; C01B 2203/1211; C07K 2319/60; C25B 1/02

(57) ABSTRACT

Compositions and methods for hydrogen production are disclosed.

### CROSS REFERENCE TO RELATED APPLICATION

This application claims priority to U.S. Provisional Application No. 63/257,464, filed on Oct. 19, 2021, the entire disclosure of which is incorporated herein by reference as though set forth in full.

### STATEMENT REGARDING GOVERNMENT FUNDING

This invention was made with government support under Grant number 80NSSC18M0093 awarded by The National Aeronautics and Space Administration. The government has certain rights in the invention.

### INCORPORATION BY REFERENCE OF MATERIAL SUBMITTED IN ELECTRONIC FORM

The contents of the electronic sequence listing (RUT-106-US.xml; Size: 24,717 bytes; and Date of Creation: Jun. 9, 2023) is herein incorporated by reference in its entirety.

### FIELD OF THE INVENTION

The present invention relates to the fields of enzyme evolution and reversible oxidation. More specifically, the invention provides peptide sequences and variants thereof capable of forming a complex with two nickel 2+ ions for catalytic generation of hydrogen under anaerobic conditions upon reduction.

### BACKGROUND OF THE INVENTION

Several publications and patent documents are cited throughout the specification in order to describe the state of the art to which this invention pertains. Each of these citations is incorporated by reference herein as though set forth in full.

Currently available methods for hydrogen production include extraction from fossil fuels such as methanol or ethanol, which depletes these resources for other applications and requires the desulfurization of petroleum products, increasing both energy and environmental costs. While direct electrolysis of water can be used to create hydrogen, in the absence of catalysts, this is an energy intensive process that is very costly. Finally, hydrogen can be produced by the pyrolysis (burning) of waste and biomass, which requires high temperatures upwards of 800° C., and generates potentially toxic side-products.

Alternatively, hydrogen can be produced using photobiological systems where photoautotrophic microorganisms are cultured in bioreactors with sunlight, and hydrogen gas is extracted. This technology is currently inefficient and subject to active research supported by the Department of Energy (DoE, https://www.energy.gov/eere/fuelcells/hydrogen-production-photobiological).

Hydrogen has many valuable commercial purposes yet current methods of production are costly, energy intensive and harmful to the environment. Clearly, improved compositions and methods for the production of hydrogen are urgently needed.

### SUMMARY OF THE INVENTION

In accordance with the invention, an isolated NB-2Ni peptide comprising at least 13 amino acids coordinating a di-nickel cluster which catalyzes production of H₂ in the presence of a suitable electron donor is disclosed. In certain embodiments, the peptide comprises any one of SEQ ID NOS: 1-12 or a sequence 90% identical thereto which is synthetic and present in an aqueous solution. In other embodiments, an isolated polynucleotide encoding at least one of the peptides described above is provided. In certain embodiments, the peptide is fused to another polypeptide of interest and is produced as a fusion protein. Also disclosed is a cell comprising the isolated polynucleotide encoding the peptide of the invention.

In another embodiment, a method of generating hydrogen is provided. An exemplary method comprises combining the peptide of the invention with an electron donor so as to generate an electron transfer chain, wherein said electron transfer chain is configured such that said electron donor is capable of donating electrons to said peptide, thereby generating hydrogen. In certain embodiments, the electron donor is selected from the group consisting of a biomolecule, a chemical, water, an electrode and a combination of the above. Preferably, the hydrogen is generated using photochemical reduction. In yet another aspect, the peptide of the invention can be used to advantage for generation of hydrogen under both anaerobic or aerobic conditions. The methods of the invention can also include the hydrogen so produced.

The invention also provides a system comprising the peptide described above and an electron donor. The electron donor can comprise an agent selected from the group consisting of a biomolecule, a chemical, water, an electrode and any combination of the above. In certain embodiments of the system, the biomolecule is comprised in particles. In other embodiments, the system can be expressed in cells.

Also provided is a bioreactor for producing hydrogen, comprising: a vessel holding a hydrogen producing system, said system comprising a suspension of the peptide described herein; a light providing apparatus comprising an LED panel or clear surface for sunlight penetration, said light providing apparatus being configured to provide light of a selected spectrum to said system; and a gas liquid separation membrane for separating gas leaving the suspension from said suspension. The vessel will include temperature and pH controlling mechanisms.

### BRIEF DESCRIPTION OF THE DRAWINGS

FIG. 1A-1F. Computationally guided design of a minimal peptide capable to coordinate a di-nickel cluster (NB-2Ni). (FIG. 1A) Modern hydrogenases are complex protein nanomachines, but their active di-metal sites (zoomed) comprise just a few amino acids. (FIG. 1B) The de novo designed NB-2Ni peptide comprised of 13 amino acids coordinating a di-nickel cluster, with the top and side views of its active site shown in (FIG. 1C). FIG. 1D-1F: Model structure of Nickelback (NB) and comparison to natural enzymes. Desulfovibrio [Ni—Fe] hydrogenase (left, PDB ID: 5XLE) and Carboxydothermus acetyl-CoA synthase (right, PDB ID: 1RU3) are large, complex proteins with active di-metal sites coordinated by a few ligands. The model structure of NB (center) combines elements of both active sites in a 13-residue polypeptide.

FIG. 2A-2F. Physicochemical characterization of Ni-reconstituted NB. (FIG. 2A) CD spectral transformations during Ni reconstitution to NB as a function of added Ni (as labeled): [NB]=750 μM, pH 7.5, temperature=50° C. The peak at 340 nm (assigned to 2NB-1Ni) at first grows reaching the maximum at [Ni]=450 μM (solid purple line), and then declines at even higher [Ni] concentrations. The peak at 430 nm (assigned to NB-2Ni) starts to develop only at [Ni]>500 μM and it grows monotonously to saturate around [Ni]=1650 μM (solid blue line). Black arrows show isosbestic points. (FIG. 2B) Fractional concentrations of 2NB-1Ni and NB-2Ni as extracted from the two-component spectral decomposition of the CD spectra from (A), as demonstrated in FIG. 3. The dashed lines are the fits using a two-step reconstitution model: apo-NB→2NB-1Ni→NB-2Ni. The upper bounds for Ni²⁺ binding constants were estimated from the fit to be around 1 μM in both 2NB-1Ni and NB-2Ni. (FIG. 2C) Absorption spectra of pure 2NB-1Ni and NB-2Ni with characteristic bands labeled. (FIG. 2D) Reduction waves in bulk solution CV experiments at different stages of Ni reconstitution in NB: [NB]=750 μM, pH 7.5, added [Ni] is indicated for each trace. The catalytic current at −850 mV (vertical dashed line) starts to develop only at [Ni]>500 μM and it grows linearly with the NB-2Ni fraction as demonstrated in (FIG. 2E). (FIG. 2F) Photochemical H₂ evolution by NB-2Ni (10 μM) driven by Eosin Y (500 μM) as photosensitizer, in presence of TEOA (500 mM) as sacrificial donors, under 540 nm LED light illumination at pH 8 and 37° C.

FIG. 3: Spectral decomposition of the CD spectra measured during a stepwise reconstitution of Ni²⁺ ions into NB as presented in FIG. 2A. The NB concentration was 750 mM, and concentrations of added Ni²⁺ as indicated on each panel. The measured CD spectra (blue traces) were decomposed as weighted sums of two spectral components, S=c₁·S₁+c₂·S₂, with an additional constraint of c₁+c₂=1 applied at Ni²⁺ concentrations higher than 450 mM. The CD spectrum measured at the lowest Ni²⁺ concentration (150 mM) was used as the basic component S₁, and its amplitude was adjusted during the fit process to account for the fact that NB was only partially reconstituted at this low Ni²⁺ concentration. The CD spectrum measured at the highest Ni²⁺ concentration (1650 mM) was taken as the basic component S₂. These two spectral components S₁ and S₂ are shown on the bottom right panel where they are labeled as 2NB-1Ni and NB-2Ni, respectively, in accordance with their structural assignments. The best decomposition fits at each Ni concentration are shown with red dashed traces. The weight coefficients (c₁ and c₂) extracted from these fits were directly used in FIG. 2B in the main text to plot relative fractions of 2NB-1Ni (c₁) and NB-2Ni (c₁) during Ni reconstitution process.

FIG. 4A-4D. Structural characterization of NB-2Ni. EPR spectra of (FIG. 4A) reduced and (FIG. 4B) oxidized NB-2Ni at pH 10, measured at 20 and 30 K, respectively: (solid lines) experiment, and (dashed lines) EPR simulations. The principal g-factor values are marked with vertical lines and numbers. The oxidized NB-2Ni in (FIG. 4B) was measured in absence (blue traces) and presence (red traces) of imidazole ligands (10 mM). FIG. 4C-4D: The g-factor symmetry of reduced (oxidized) NB-2Ni is similar to Ni¹⁺ in [NiFe]-hydrogenase (blue dashed circle) and Ni³⁺ in Ni-SOD (red dashed circle), where Ni ions are found in distinctly different coordination sites, e.g. Ni¹⁺(S₄Xₙ) and Ni³⁺(S₂N₂), respectively. Both these Ni sites are represented in di-nickel NB-2Ni, with one (proximal) Nip site and the second (distal) Ni_{d} site.

FIG. 5 shows H₂ production using photochemical reduction using light as the electron source.

FIG. 6A-6B: Initial stages of NB design. (FIG. 6A) the Mn binding site of parvalbumin (PDB ID: 2PAL) was used as a template for engineering a nickel coordination site. (FIG. 6B) Molecular model of a mononuclear Ni-peptide complex sampled by molecular mechanics simulations.

FIG. 7: DFT geometry optimized structures of CGC and CNC models of NB-2Ni and full structures with outline indicating solvent-excluded surface. Highlighting key ligand residues (C=cysteine, N=asparagine, R=arginine, G=glycine). Of these, the CGC model is most consistent with EPR measurements.

FIG. 8: CD spectral transformation in the course of Ni²⁺ reconstitution into NB. The Ni²⁺ reconstitution into 750 μM of NB was performed at 50° C. in 25 mM HEPES, pH7.5, NaCl 100 mM, 3.75 mM TCEP, by gradually adding Ni²⁺ to concentration indicated for each curve. The spectra revealed complex transformations with (1) the peak at 334 nm growing up at first at low Ni²⁺ concentration 0-450 μM, and then declining down at higher concentrations 450-1650 μM, and (2) the peak at 428 nm starting to grow only at Ni²⁺ concentrations higher than 450 μM. Black arrows indicate isosbestic points in these spectral transformations. These CD spectra were next decomposed into two spectral components corresponding to two species: (1) the first assembly intermediate 2NB-1Ni, and (2) the final assembly product NB-2Ni, as shown in FIG. 3.

FIG. 9A-9F: Dynamic Light Scattering measurements of NB-2Ni with controls. The measurements were carried out in 50 mM HEPES pH 7.5, 100 mM NaCl and 5 mM TCEP, after 2 days incubation at room temperature under nitrogen atmosphere. The reconstitutions of NB-2Ni and 4Cys-2Ni were monitored by CD spectroscopy. Buffer only control: (FIG. 9A) size distribution by mass, (FIG. 9B) correlogram. 4Cys-2Ni control: (FIG. 9C) size distribution by mass, (FIG. 9D) correlogram with repeats in red and green does not converge at a coefficient of 1 near time zero, indicating a poor fit to a discrete set of species. NB-2Ni: (FIG. 9E) size distribution by mass, (FIG. 9F) correlogram with repeats measured in red, green and blue, displaying clear presence of a particle species of 1.29±0.22 nm. Additional species are resolved but are a very minor fraction of the total species.

FIG. 10: pH stability of fully assembled NB-2Ni as monitored by CD spectroscopy. The [NB-2Ni]=530 μM in the four-component buffers, including 10 mM potassium phosphate, 10 mM MES, 10 mM HEPES, and 10 mM CHES, NaCl 100 mM, and 3.75 mM TCEP. The CD experiments were performed at room temperature. pH values are indicated for each curve.

FIG. 11: Temperature stability of fully assembled NB-Trp-2Ni as monitored by CD spectra. The [NB-Trp-2Ni]=660 μM in 25 mM HEPES, pH7.6, NaCl 100 mM and 3.75 mM TCEP. The CD experiments were performed in the temperature range 20-90° C. as indicated.

FIG. 12: Oxygen sensitivity of fully-assembled NB-2Ni as monitored by CD spectra. The [NB-2Ni]=300 μM in 25 mM HEPES, pH7.6, NaCl 100 mM and 3.75 mM TCEP. The CD spectra were measured at room temperature before (blue curve) and after (other colors) purging the NB-2Ni solution with O₂ gas for the indicated periods of time.

FIG. 13A-13B: Reduction waves from CV experiments after their baseline correction. Two scan rates were used: (FIG. 13A) 500 mV/s, and (FIG. 13B) 50 mV/s. The reduction waves at −850 mV, and the oxidation wave at −770 mV (not shown), started to develop only at high concentrations of added Ni²⁺ greater than 500 μM. This led us to conclude that the assembly intermediates, 2NB-1Ni, is redox inactive, and the redox waves belong to the fully assembled NB-2Ni. This assignment was further strengthened by observing a linear correlation between the maximum peak current at −850 mV in CV experiments and the fractional concentration of NB-2Ni as derived from our CD analysis and shown in FIG. 2E.

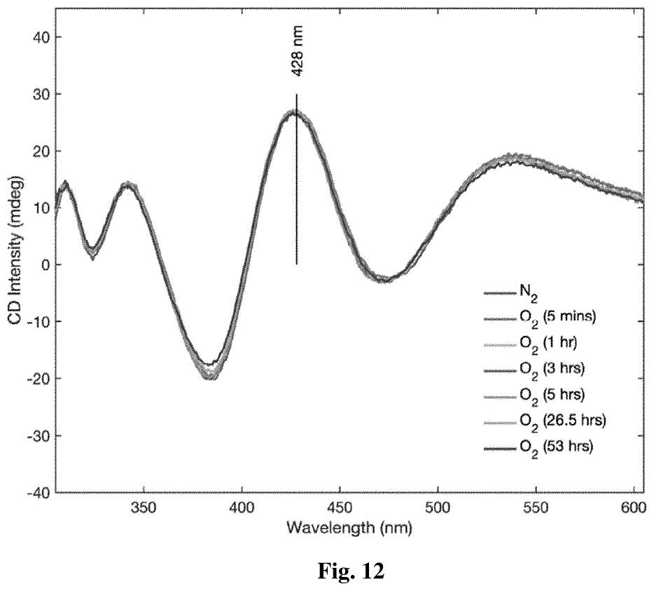

FIG. 14: CV traces for three NB variants. All variants were fully reconstituted with Ni²⁺ ions at 50° C. in 25 mM HEPES, pH7.5, with 100 mM NaCl and 5 mM TCEP. The CV measurements were performed at room temperature with the scan rate 500 mV/s. Notice a slight difference in NB's concentrations as indicated in the curve legends. Two variants, NB-2Ni and NB-Trp-2Ni, demonstrated redox activity with reduction potential peaking around −840±5 mV. These two variants were reconstituted with two Ni²⁺ per peptide: one at proximal Ni(S₄) and the second at distal Ni(N₂S₂) positions.

FIG. 15A-15B: CD and CV traces for NB-2Ni compared to L-cysteine-Ni. The specificity of our NB-2Ni complex is supported by the clear distinction of our peptide complex from a poorly defined Ni-thiol complex. For this, NB peptide and L-cysteine were reconstituted in 50 mM HEPES, pH 7.5, 100 mM NaCl and 5 mM TCEP at room temperature for 2 days under nitrogen atmosphere. For the experiment 0.75 mM NB were used with 1.5 mM NiCl₂ (NB-2Ni) and 3 mM L-cysteine with 1.5 mM NiCl₂ (4Cys-2Ni), to keep an equimolar concentration of cysteines and nickel in both samples. (FIG. 15A) shows the CD spectra recorded at room temperature with NB-2Ni in blue and 4Cys-2Ni in yellow. These samples were analyzed using Circular voltammetry with a scan rate of 500 mV/s at room temperature (FIG. 15B). The NB-2Ni displayed redox activity with reduction potential peaking around −840±5 mV, while L-cysteine-Ni did not.

FIG. 16: Hydrogen flux estimation from photochemical excitation of NB-2Ni for a 1 Liter volume. We wish to estimate here whether small metallo-peptides, like NB-2Ni, through their photocatalytic activity, could potentially generate large concentrations of dissolved H₂ in water at the level that is sufficient to fuel up the initial (prebiotic) evolution of H₂-dependent metabolic reactions.

FIG. 17: CD spectra of three variants of NB peptide. All variants were reconstituted with Ni2+ ions at 50° C. in 25 mM HEPES, pH7.6, with 100 mM NaCl. The CD spectra were measured at room temperature. Peptide concentrations indicated in the figure legend. Three NB variants, NB-2Ni and NB-Trp-2Ni, showed remarkably similar CD spectra, both in terms of peak positions and peak intensities. This similarity led us to conclude that: (1) these variants maintained the same stoichiometry of two bound Ni per peptide, and (2) the coordination geometry of the two Ni²⁺ sites was quite similar, e.g. one proximal Ni(S₄) site and the second distal Ni(S₂N₂) site. This conclusion was further supported in UV-visible absorption experiments, cyclic voltammetry, and also EPR. On the other hand, the NBDP-Ni variant displayed a distinctly different CD spectrum (yellow curve). This variant had two out of four cysteines mutated to serines, and therefore only one binding site for Ni²⁺ at the distal position Ni(S₂N₂). Coordination of a single Ni²⁺ in NBDP-Ni was further confirmed in our EPR experiments.

FIG. 18: Exemplary electrochemical cells including a classic electrolysis cell, a zero-gap electrolysis cell, an asymmetric PEM electrolysis cell, and a capillary fed electrolysis cell. (Hodges, A. et al. A high-performance capillary-fed electrolysis cell promises more cost-competitive renewable hydrogen. Nat Commun 13, 1304 (2022).

FIG. 19: Exemplary photoelectrochemical cell.

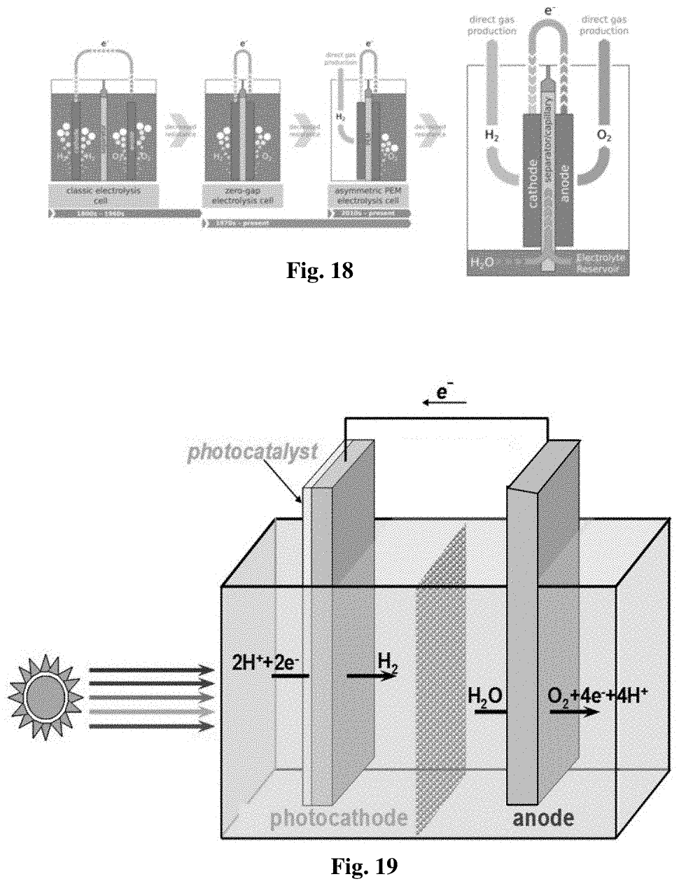

FIG. 20: Exemplary Microbial Fuel Cell. Electroactive bacteria are present on the anode to drive electron production. The peptides presented herein, or bacteria comprising said peptides, are present on the cathode to catalyze the reaction producing H₂.

### DETAILED DESCRIPTION OF THE INVENTION

One of the most ancient processes for energy production in the evolution of life is the reversible oxidation of molecular hydrogen by hydrogenase. Extant hydrogenase enzymes are complex and comprised of more than 400 amino acids, several subunits, and multiple cofactors, which makes production in large quantities difficult. As described herein below, we have designed and synthesized a 13 amino acid nickel-binding peptide which robustly produces molecular hydrogen from protons under a wide variety of conditions. The peptide forms a di-nickel cluster structurally analogous to a Ni—Fe cluster in [NiFe]-hydrogenase and a Ni—Ni cluster in Acetyl-CoA synthase, two ancient proteins central to metabolism in all life extant forms.

As demonstrated herein, very short peptides can readily form di-nuclear nickel clusters capable of catalytically evolving H₂. Such peptides are simple enough that they could have emerged spontaneously during a prebiotic stage in the origin of life, giving rise to the first biotic metabolisms.

Unless otherwise defined herein, scientific and technical terms used in connection with the present application shall have the meanings that are commonly understood by those of ordinary skill in the art. In addition to definitions included in this sub-section, further definitions of terms are interspersed throughout the text.

In this invention, “a” or “an” means “at least one” or “one or more,” etc., unless clearly indicated otherwise by context. The term “or” means “and/or” unless stated otherwise. In the case of a multiple-dependent claim, however, use of the term “or” refers back to more than one preceding claim in the alternative only.

Furthermore, a compound “selected from the group consisting of” refers to one or more of the compounds in the list that follows, including mixtures (i.e. combinations) of two or more of the compounds. According to the present invention, an isolated, or biologically pure molecule is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The terms “agent” and “test compound” denote a chemical compound, a mixture of chemical compounds, a biological macromolecule, or an extract made from biological materials such as bacteria, plants, fungi, or animal (particularly mammalian) cells or tissues. Biological macromolecules include siRNA, shRNA, antisense oligonucleotides, peptides, peptide/DNA complexes, and any nucleic acid-based molecule which encoded the proteins described herein.

It is also contemplated that the term “compound” or “compounds” refers to the compounds discussed herein and includes precursors and derivatives of the compounds, and pharmaceutically acceptable salts of the compounds, precursors, and derivatives.

The phrase “consisting essentially of” when referring to a particular nucleotide or amino acid means a sequence having the properties of a given SEQ ID NO. For example, when used in reference to an amino acid sequence, the phrase includes the sequence per se and molecular modifications that would not affect the functional and novel characteristics of the sequence.

A “derivative” of a polypeptide, polynucleotide or fragments thereof means a sequence modified by varying the sequence of the construct, e.g. by manipulation of the nucleic acid encoding the protein or by altering the protein itself. “Derivatives” of a gene or nucleotide sequence refers to any isolated nucleic acid molecule that contains significant sequence similarity to the gene or nucleotide sequence or a part thereof. In addition, “derivatives” include such isolated nucleic acids containing modified nucleotides or mimetics of naturally-occurring nucleotides.

The term “functional” as used herein implies that the nucleic or amino acid sequence is functional for the recited assay or purpose.

For purposes of the invention, “nucleic acid”, “nucleotide sequence” or a “nucleic acid molecule” as used herein refers to any DNA or RNA molecule, either single or double stranded and, if single stranded, the molecule of its complementary sequence in either linear or circular form. In discussing nucleic acid molecules, a sequence or structure of a particular nucleic acid molecule may be described herein according to the normal convention of providing the sequence in the 5′ to 3′ direction. With reference to nucleic acids of the invention, the term “isolated nucleic acid” is sometimes used. This term, when applied to DNA, refers to a DNA molecule that is separated from sequences with which it is immediately contiguous in the naturally occurring genome of the organism in which it originated. For example, an “isolated nucleic acid” may comprise a DNA molecule inserted into a vector, such as a plasmid or virus vector, or integrated into the genomic DNA of a prokaryotic or eukaryotic cell or host organism. Alternatively, this term may refer to a DNA that has been sufficiently separated from (e.g., substantially free of) other cellular components with which it would naturally be associated. “Isolated” is not meant to exclude artificial or synthetic mixtures with other compounds or materials, or the presence of impurities that do not interfere with the fundamental activity, and that may be present, for example, due to incomplete purification. When applied to RNA, the term “isolated nucleic acid” refers primarily to an RNA molecule encoded by an isolated DNA molecule as defined above. Alternatively, the term may refer to an RNA molecule that has been sufficiently separated from other nucleic acids with which it would be associated in its natural state (i.e., in cells or tissues). An isolated nucleic acid (either DNA or RNA) may further represent a molecule produced directly by biological or synthetic means and separated from other components present during its production.

A “specific binding pair” comprises a specific binding member (sbm) and a binding partner (bp) which have a particular specificity for each other and which in normal conditions bind to each other in preference to other molecules. Examples of specific binding pairs are antigens and antibodies, ligands and receptors and complementary nucleotide sequences. The skilled person is aware of many other examples. Further, the term “specific binding pair” is also applicable where either or both of the specific binding member and the binding partner comprise a part of a large molecule. In embodiments in which the specific binding pair comprises nucleic acid sequences, they will be of a length to hybridize to each other under conditions of the assay, preferably greater than 10 nucleotides long, more preferably greater than 15 or 20 nucleotides long.

Regarding the polypeptides disclosed herein, the phrases “% sequence identity,” “percent identity,” or “% identity” refer to the percentage of residue matches between at least two amino acid sequences aligned using a standardized algorithm. Methods of amino acid sequence alignment are well-known. Some alignment methods take into account conservative amino acid substitutions. Such conservative substitutions generally preserve the charge and hydrophobicity at the site of substitution, thus preserving the structure (and therefore function) of the polypeptide. Percent identity for amino acid sequences may be determined as understood in the art. The structural similarity is typically at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity.

Polypeptide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, may be used to describe a length over which percentage identity may be measured.

According to the present invention, an isolated or biologically pure molecule or cell is a compound that has been removed from its natural milieu. As such, “isolated” and “biologically pure” do not necessarily reflect the extent to which the compound has been purified. An isolated compound of the present invention can be obtained from its natural source, can be produced using laboratory synthetic techniques or can be produced by any such chemical synthetic route.

The term “delivery” as used herein refers to the introduction of foreign molecule (i.e., miRNA encoding the polypeptide of interest) into cells. The term “administration” as used herein means the introduction of a foreign molecule into a cell. The term is intended to be synonymous with the term “delivery”.

### Peptides

The peptides of the invention have hydrogenase activity. Further, the peptide of the invention binds 2 Ni2+ ions. Reconstitution kinetic is pH and temperature dependent and tolerant to various pHs and temperatures. The peptide has a complex active site most likely harboring 2 Ni2+ ions in the same geometry as Acetyl-CoA synthase (ACS), one site similar to Ni-superoxide dismutase (Ni-SOD), and can effectively transfer electrons. It exhibits an onset potential −1.05 V (Ag/AgCl2 reference) and is also tolerant of CO₂/bicarbonate. The peptide exhibits robust H₂ production: TON>500 per peptide complex.

The peptide and variants thereof contain the sequence (Z)₀₋₅—C—X—C—Y—C*—Y—(X)₁₋₅—C*—Y—(Z)₀₋₅, (SEQ ID NO: 1) with X, Y and Z representing any amino acid of the L- or D-configuration. Artificial amino acids may be used as well and include chemically activated amino acids. Y is preferably glycine but other amino acids are possible. C* can be in either L- or D-configuration. For example, small letters mean D-amino acids. N=L-Asparagine, n=D-asparagine.

Exemplary sequences include, without limitation:

(SEQ ID NO: 2)
CNCGCGNNNDRCG
(SEQ ID NO: 3)
CNCGCGNWNDRCG
(SEQ ID NO: 4)
CNCACGNWNDRCG
(SEQ ID NO: 5)
GCNCGCGNNNDRCG
(SEQ ID NO: 6)
CNCGCGNNNNNDRCG
(SEQ ID NO: 7)
GGCNCGCGNWNDRCGG
(SEQ ID NO: 8)
CNCGCGnnnDRCG
(SEQ ID NO: 9)
CGCGCGWHGGRCG
(SEQ ID NO: 10)
CRCWchcG
(SEQ ID NO: 11)
CHCWchcG

and

C—X—C—Y—C—Y—(X)₁₋₅—R—C—Y, with X and Y representing any amino acid of the L- or D-configuration, where Y is preferably glycine (SEQ ID NO: 12).

In certain embodiments, the present invention includes peptides retaining activity that have at least 80% identity to anyone of the peptides described herein. In certain embodiments, the peptides of the invention have a sequence identity of at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, at least 99% identity, or 100% identity.

Preferably, the peptides belonging to the above-described sequences and functional equivalents thereof. As used herein, the term “functional equivalent” is intended to include amino acid sequence variants having amino acid substitutions in some or all of the proteins, or amino acid additions or deletions in some of the proteins. The amino acid substitutions are preferably conservative substitutions. Examples of the conservative substitutions of naturally occurring amino acids are as follow: aliphatic amino acids (Gly, Ala, and Pro), hydrophobic amino acids (Ile, Leu, and Val), aromatic amino acids (Phe, Tyr, and Trp), acidic amino acids (Asp, and Glu), basic amino acids (His, Lys, Arg, Gln, and Asn), and sulfur-containing amino acids (Cys, and Met). The deletions of amino acids are preferably located in a region which is not directly involved in the activity of the peptide.

The peptide may be fused to biotin, fluorescein, Eosin Y, other fluorescent small molecules, Poly-lysine, lysozyme, Green fluorescent protein (and derivatives such as Orange fluorescent protein (OFP), mOrange, etc.), SUMO, ferredoxin, cytochromes, or other desired proteinaceous tags for attachment to electrodes, nanotubes nanoparticles or desired surfaces (e.g. electro-transducing, mineral), as well as any protein interaction partner desired to be investigated in terms of electron transfer properties.

Production of the desired peptide sequence can be carried out in E. coli using existing technologies, e.g. with protein fusion tags that can either be removed or left as desired.

Advantageously, the peptide has very few fixed residues—specifically four cysteines required for assembly of the di-nickel active site. This allows significant flexibility in engineering variant sequences for specific applications, such as tuning redox potential by vary charge, peptide hydrophobicity for targeting nonpolar electroactive surfaces (graphene, carbon nanotubes), and altering the reactivity to different gasses.

The peptide can be expressed as a fusion to larger proteins, facilitating expression at large scales, ease of purification, and ensuring quality of product. Expression systems can also be leveraged to generate large sequence libraries, allowing for directed evolution for targeted properties. Catalysts can be produced sustainably using environmentally-friendly, existing fermentation technologies. Fusion also allows the catalyst to be incorporated into other enzymes to promote multi-step chemical reactions or to function within biological electron transport chains.

In certain embodiments, the peptide can be fused to any protein involved in photosynthesis. In preferred embodiments of the invention, the c-peptides can be directly fused to at least one of photosystems PS1 and PS2 for direct coupling of hydrogen evolution and light harvesting.

Exemplary fusion proteins are provided below:

KBtryp_Kbio1
(SEQ ID NO: 14)
CNCGCGNWNDRCGGK[biotin]
NBtryp_Kbio2
(SEQ ID NO: 15)
CNCGCGNWNDRCGGGK[biotin]

Both peptides bind Nickel ions in the same way than the unfused peptide. The electronic behavior is identical with an onset potential of −1.05V against Ag/AgCl electrode.

Robust binding of the biotin-peptide-Ni complex to a protein (short Hoefly Avidin) while maintaining the Nickel coordination was shown. This indicates that a bulky modification of the peptide is possible without impacting the nickel-coordination.

As noted above, the invention also includes polynucleotides encoding the peptides or fusion proteins comprising the peptide described herein. Those of skill in the art understand the degeneracy of the genetic code and that a variety of polynucleotides can encode the same polypeptide. In some embodiments, the polynucleotides (i.e., polynucleotides encoding the fusion polypeptides) may be codon-optimized for expression in a particular cell including, without limitation, a plant cell, bacterial cell, cyanobacterial cell, archaeal cell, fungal cell, or algal cell. Any polynucleotide sequences may be used which encode a desired form of the polypeptides described herein. The polynucleotide sequences which encode the polypeptides of the invention represent non-naturally occurring sequences. Computer programs for generating degenerate coding sequences are available and can be used for this purpose. Pencil, paper, the genetic code, and a human hand can also be used to generate degenerate coding sequences.

Transgenic cells expression said polynucleotides also form an aspect of the invention. A transgenic cell may be obtained by introducing a recombinant nucleic acid molecule that encodes a protein of this disclosure. As used herein, the term “recombinant nucleic acid” refers to a polynucleotide that is manipulated by human intervention. A recombinant nucleic acid molecule can contain two or more nucleotide sequences that are linked in a manner such that the product is not found in a cell in nature. In particular, the two or more nucleotide sequences can be operatively linked and, for example, can encode a fusion polypeptide. A recombinant nucleic acid molecule also can be based on, but manipulated so as to be different, from a naturally occurring polynucleotide, for example, a polynucleotide having one or more nucleotide changes such that a first codon, which normally is found in the polynucleotide, is biased for chloroplast codon usage, or such that a sequence of interest is introduced into the polynucleotide, for example, a restriction endonuclease recognition site or a splice site, a promoter, a DNA origin of replication, or the like.

Any appropriate technique for introducing recombinant nucleic acid molecules into cells may be used. Techniques for nuclear and chloroplast transformation are known and include, without limitation, electroporation, biolistic transformation (also referred to as micro-projectile/particle bombardment), agitation in the presence of glass beads, and Agrobacterium-based transformation. In certain embodiments, the recombinant nucleic acid molecules are introduced into cells using CRISPR-Cas9 systems. Use of CRISPR-Cas9 is known by the skilled artisan. See, e.g., Adli, M. The CRISPR tool kit for genome editing and beyond. Nat Commun 9, 1911 (2018).

As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies.

A “vector” is capable of transferring gene sequences to target cells. Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as integrating vectors.

The constructs and vectors provided herein may be prepared by methods available to those of skill in the art. Notably each of the constructs or expression cassettes claimed are recombinant molecules and as such do not occur in nature. Generally, the nomenclature used herein and the laboratory procedures utilized in the present invention include molecular, biochemical, and recombinant DNA techniques that are well known and commonly employed in the art. Standard techniques available to those skilled in the art may be used for cloning, DNA and RNA isolation, amplification and purification. Such techniques are thoroughly explained in the literature.

The constructs and expression cassettes provided herein may include a promoter operably linked to any one of the polynucleotides described herein but need not have a promoter and may be used for homologous recombination into the cell. Alternatively, the constructs may include a promoter and the promoter may be a heterologous promoter or an endogenous promoter associated with the polypeptide.

As used herein, the terms “heterologous promoter,” “promoter,” “promoter region,” or “promoter sequence” refer generally to transcriptional regulatory regions of a gene, which may be found at the 5′ or 3′ side of the polynucleotides described herein, or within the coding region of the polynucleotides, or within introns in the polynucleotides. Typically, a promoter is a DNA regulatory region capable of binding RNA polymerase in a cell and initiating transcription of a downstream (3′ direction) coding sequence. The typical 5′ promoter sequence is bounded at its 3′ terminus by the transcription initiation site and extends upstream (5′ direction) to include the minimum number of bases or elements necessary to initiate transcription at levels detectable above background. Within the promoter sequence is a transcription initiation site (conveniently defined by mapping with nuclease S1), as well as protein binding domains (consensus sequences) responsible for the binding of RNA polymerase.

In some embodiments, the disclosed polynucleotides are operably connected to the promoter. As used herein, a polynucleotide is “operably connected” or “operably linked” when it is placed into a functional relationship with a second polynucleotide sequence. For instance, a promoter is operably linked to a polynucleotide if the promoter is connected to the polynucleotide such that it may affect transcription of the polynucleotides. In various embodiments, the polynucleotides may be operably linked to at least 1, at least 2, at least 3, at least 4, at least 5, or at least 10 promoters.

Heterologous promoters useful in the practice of the present invention include, but are not limited to, constitutive, inducible, temporally-regulated, developmentally regulated, chemically regulated, tissue-preferred and tissue-specific promoters. The heterologous promoter may be a plant, animal, bacterial, fungal, or synthetic promoter.

In some embodiments, the polynucleotides are fused endogenous genes of a light harvesting protein, like PS1 and PS2, or an endogenous electron transfer protein, like ferredoxin.

### Methods of Use

In another aspect, provided herein are methods for producing hydrogen. H₂ is an important feedstock in the chemical industry for producing ammonia, methanol and other chemicals, as well as a clean source of energy that is already being explored by the transportation sector for its low emissions.

Provided herein are methods for using a polypeptide having hydrogenase activity. The methods may include providing a polypeptide described herein, and incubating the polypeptide under conditions suitable for producing H₂. The produced H2 may be collected.

As used herein, “hydrogenase activity” refers to the ability of a polypeptide to catalyze the formation of molecular hydrogen (H₂). The peptides described herein can catalyze the following reversible reaction which creates hydrogen gas from protons:

2H⁺+2e⁻↔H₂

In one aspect, the polypeptide is an isolated or purified polypeptide. The polypeptide may be present on a surface, such as one that conducts electricity, e.g., an anode. The polypeptide may be chemically modified. The incubating may include conditions that include a polysaccharide, such as a starch or a cellulose. The incubating may include nanoparticles or nanotubes. The conditions for incubating the peptides with carbon nanoparticles and nanotubes are known to the skilled artisan. See, e.g., D. Baskaran et al. Carbon Nanotubes with Covalently Linked Porphyrin Antennae: Photoinduced Electron Transfer. J. Am. Chem. Soc. 2005, 127, 19, 6916-6917 The conditions can include a temperature of at least 37° C., or at least 70° C. The peptide is stable at a range of temperatures from 4-90° C., at a range of pH from acidic to basic, and for a number of aqueous buffer conditions, allowing solution conditions to be optimized to accommodate other components in a hydrogen-production system.

In another aspect, the polypeptide is present in a genetically modified microbe. The incubating may include incubating the microbial cell under conditions suitable for the expression of the polypeptide. To produce hydrogen, the genetically engineered cells described herein can be cultured in a bioreactor growth system and the gas released during growth can be collected, removed from the bioreactor, and the hydrogen can be separated and collected from the remaining air in the bioreactor after growth of the cells. The cells expressing the peptides of the invention described herein may be cultured under conditions and in media to increase H₂ production. For example, the cells may be cultured under saturating light conditions to induce increased H₂ production.

In some embodiments, it will be advantageous to measure hydrogenase activity. Any appropriate means of measuring hydrogenase activity may be used. In some embodiments, samples are collected for analysis of hydrogenase activity by gas chromatography (GC). Those of skill in the art are aware of methods to measure hydrogenase activity and such methods are provided in the Examples.

Further commercial applications of the nickel-binding peptide hydrogenase include, without limitation:

(1) Use as an electrocatalyst that can be fused to electrodes or other electroactive materials, either noncovalently or through chemical linkers. Solar or other renewable energy can be used to sustainably produce H₂ from water.

(2) A photocatalyst that can be coupled with photosensitizers to promote direct light-driven hydrogen production. This has already been demonstrated to work with the organic dye eosin Y or with titanium oxide nanoparticles as photosensitizers.

(3) A component in microbial fuel cells that will be expressed separately, or as a fusion with an electron donating protein to produce molecular hydrogen within the cell.

The small size of the peptide (13 residues) allows it to be incorporated at high densities on electrodes or other electroactive materials, facilitating scale-up of hydrogen production.

Finally, the peptide is redox-stable, with turnover numbers >500.

### Bioreactors

In certain embodiments, the peptides of this invention may be included in a bioreactor. Bioreactors are vessels or tanks in which whole cells or cell-free enzymes transform raw materials into biochemical products and/or less undesirable by-products. The term “bioreactor” refers to a biological reactor, and is either a bioreaction vessel, or a bioreaction enclosure, or a bioreaction tank, and/or at least a bioreaction chamber, and/or a cell, or a combination thereof. Bioreactors are able to withstand variations of temperature and/or pressure, among others, and/or able to maintain whichever imparted values of temperature, and/or pressure are assigned or have to be maintained, before, after or during the reaction process, and wherein the intended reactions relevant for carrying out the invention may take place.

Such reactions are understood as bioreactions as they pertain to the domain of reactions wherein microorganisms are involved, and herein referring to their normal physiology—such as hydrogen production, metabolic fermentation, or aerobic or anaerobic digestion—and that, as such, require suitable environments, suitable cultures of microorganisms, suitable culture mediums and suitable reactants to occur. A bioreactor in the meaning of the invention, performs reliably within the tolerance values of each variable in order to enable the method as disclosed, and it is expected to allow the listed steps to be carried out reliably overtime.

In certain embodiments, the bioreactor is capable of at least one of pH control, temperature control, stirring/mixing, aeration, a feeding outlet for introducing a nutrient media, a waste outlet, and a gas separation membrane for hydrogen harvesting.

Suitable reactors for producing hydrogen include, without limitation, a shake tank bioreactor, a continuous stirred tank bioreactor, an intermittent stirred tank bioreactor, a hollow fiber membrane bioreactor, a bubble column bioreactor, a microbubble reactor, an airlift bioreactor an internal-loop airlift bioreactor, an external-loop airlift bioreactor, a fluidized bed bioreactor, a packed bed bioreactor, a photo-bioreactor, a trickle bed reactor, a microbial electrolysis cell, and/or combinations thereof. Bioreactors may be used where the peptides are present as electrocatalysts, as photocatalysts, and/or in microbial fuel cells.

In still another aspect, the present disclosure provides a bioreactor for cultivating cells containing the peptides provided herein. The present bioreactor includes at least one reactor vessel. The reactor vessel may be vessels that can be used as a bioreactor for cultivating cells. The vessel has any appropriate shape, including for example, planer (such as flat panel), tubular (such as tube, hose or pipe), cylindrical (such as tank), bottle-shaped, dome-shaped, cuboid, polyhedron and others. The vessel may be a tubular vessel folded in a meandering shape. The vessel may be a plastic film tube. The reactor vessel may be formed using a glass or plastic with high visible light transmittance (such as acrylic resin, polyethylene, polypropylene, polycarbonate, polystyrene, polyvinyl chloride, nylon or the like). The vessel may have any appropriate size. An appropriate size can be determined according to the desired scale of the cultivation.

The reactor vessel has a first opening, through which the cells and the cultivation medium can be introduced in the vessel and the culture can be collected from the vessel. The first opening may be configured to be sealed after introducing the cells and the cultivation medium.

The reactor vessel may have a second opening to collect hydrogen produced in the bioreactor. The first opening may be used also for hydrogen collection.

The reactor vessel may be provided with a stirrer capable of stirring the cells and the cultivation medium contained therein.

The present bioreactor may include at least one lighting device. When the lighting device is present, the bioreactor is a photobioreactor. The present photobioreactor may include a single lighting device combined with one or more reactor vessels, or two or more lighting device combined with one or more reactor vessels.

The lighting device emits light (artificial light) having a ratio of (i) photon flux density in the wavelength range of 520-630 nm to (ii) photosynthetic photon flux density, that is 65% or more, more particularly 70% or more, more particularly 75% or more, more particularly 80% or more, more particularly 85% or more, more particularly 90% or more, and more particularly 95% or more. In the context of the present photobioreactor, the “photon flux density” is the one measured at the inner or outer surface (preferably, outer surface) of the reactor vessel to be irradiated with light from the lighting device (i.e., the artificial light). The lighting device may include a single light source, or two or more light sources.

In some preferred embodiments, the lighting device has such a light-emitting device (LED) or laser diode (LD) that emits light. In this case, the lighting device may be provided as a cluster or an array of LDs or LEDs. The embodiments enable to suppress the radiation of red light and/or blue light that can have an adverse effect on the cells (such as cell damage by active oxygen species generated) at too high intensity. The embodiments also allow efficient irradiation with such light capable of reaching the cells in the central part and more distant parts (along the irradiation axis) of the cultivation medium containing the cells, which are contained in the reactor vessel, and of being absorbed (and utilized for photosynthesis) by chlorophylls. In view of energy efficiency and economic efficiency, use of LED or LD is preferable due to the energy intensiveness, low heat generation, low power consumption and long life. In addition, the photon flux density can be easily controlled and maintained. In certain embodiments, the photobioreactor includes a control unit to control the light source.

In certain embodiments, the bioreactor further comprises a sensor configured to measure a condition(s) of the cells and/or the cell culture in the reactor vessel.

Bioreactors are known by the skilled artisan and readily available. See, e.g., Photo Systems Instruments (PSI.cz) available on the world wide web at photo-bio-reactors.com/documents/PBR_list_of_references.pdf

### Peptides as an Electrocatalyst

Most of the hydrogen produced by electrolysis comes from electrolytic water splitting and the chloralkali processes, which require efficient and stable hydrogen evolution reaction (HER) electrocatalysts. In certain embodiments, the peptides described herein can be used as electrocatalysts.

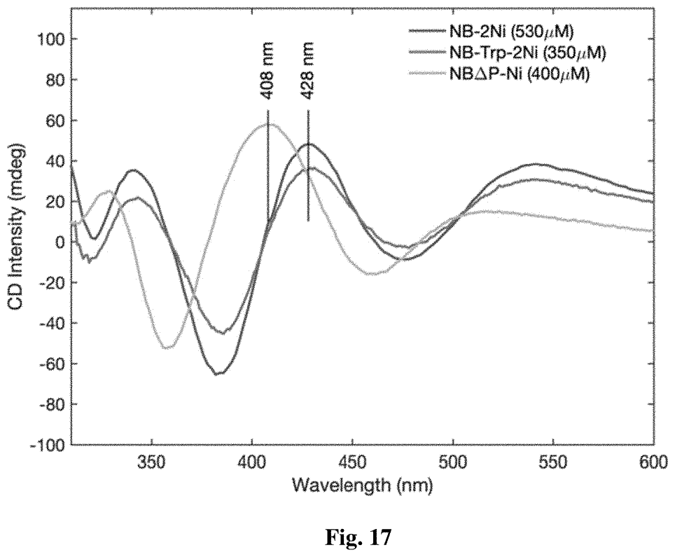

Generally, electrolysis occurs in an electrochemical cell. An electrochemical cell contains an anode, a cathode, and an electrolyte. Electrocatalysts are placed on the anode, and/or the cathode, and/or in the electrolyte to promote the desired chemical reactions. During operation, reactants or a solution containing reactants is fed into the cell. Then a voltage is applied between the anode and the cathode, to promote an electrochemical reaction. As used herein, the electrochemical reaction produces H₂. Electrochemical cells are well known to the skilled artisan. See, e.g., Wang, S., Lu, A. & Zhong, CJ. Hydrogen production from water electrolysis: role of catalysts. Nano Convergence 8, 4 (2021). An exemplary electrochemical cell is provided herein as FIG. 18. Electrochemical cells may be incorporated into a bioreactor.

For the purposes of the present invention, the terms “electrocatalyst” and “electrode” are used as equivalent and interchangeable synonyms. An electrocatalyst is a catalyst that participates in an electrochemical reaction (i.e. functioning at electrode surfaces or being the electrode surface itself) by modifying and increasing the rate of the reaction without being consumed in the process. See e.g. Xu, Yong and Schoonen, Martin A. A. “The absolute energy positions of conduction and valence bands of selected semiconducting minerals” American Mineralogist, vol. 85, no. 3-4, 2000, pp. 543-556

Preferably the electrocatalyst of the invention does not undergo degradation in such conditions and it can be used several times without the need to be regenerated.

In order to bind the peptide to an electrode or electron-donating nanomaterial, the peptide can be modified to improve binding to those surfaces. Those modifications include poly-lysine and/or biotin modifications. Exemplary electron-donating materials include, without limitation, Titanium oxide (TiO) nanoparticles, electron-donating nanotubes, hydrogen-storing nanomaterials, and glassy carbon.

### Peptides as Photocatalysts

The production of hydrogen with visible light is a promising pathway using hydrogen protons and sunlight i.e., an abundant raw material and limited energy source on human scale. This method is therefore of major economic and environmental interest.

In order to be activated by sunlight, a light harvesting peptide or chemical must be present. The light harvesting the chemical or peptide will harvest the light and transfer the electrons to the peptide. In certain embodiments, the photocatalysts are fusion proteins comprising the peptides disclosed herein fused to a light-harvesting protein, like PS1 and PS2, green fluorescent protein, orange fluorescent protein and derivatives, or a photo-activated chemical, like Eosine Y, Fluorescein. In certain embodiments, the light-harvesting proteins or chemicals are covalently added to either terminus of the peptide via an activated amino acid side chain.

Alternatively, the peptide can be fused to ferredoxin, which obtains the electrons from the photosystems inside cells, and then in turn relays them to the peptide for hydrogen production. The term “photocatalyst” refers to a material which absorbs light to bring it to higher energy level and provides such energy to a reacting substance to make a chemical reaction occur. As used herein, the chemical reaction produces H₂. The absorption range of a photocatalysts can include anywhere in the electromagnetic spectrum. In preferred embodiments, the photocatalyst has an absorption range which includes visible light, UV light, and/or infrared light. In certain embodiments the photocatalyst does not have an absorption range which outside of visible light, UV light, and/or infrared light.

Photoelectrochemical cells are well known to the skilled artisan. See, e.g Yi-Hsuan C. et al., “Photoelectrochemical cells for solar hydrogen production: Challenges and opportunities”, APL Materials 7, 080901 (2019); and K. C. Christoforidis et al., Photocatalytic Hydroben Production: A Rift into the Future Energy Supply, ChemCatChem 2017, 9, 1523. An exemplary photoelectrochemical cell is provided herein as Figure YY. Photoelectrochemical cells may be incorporated into a bioreactor.

### Peptides in Fuel Cells

A microbial fuel cell (MFC) refers to an apparatus which can directly convert chemical energy of organic matter into electric energy using microbes capable of extracellularly transferring electrons, among anaerobic microbes feeding on organic matter. Hydrogen can then be generated by applying the electric current produced by the cell to the cathode.

The microbial fuel cell described herein provides a bio-electro-chemical system capable of generating electricity, comprising: an anode chamber housing an anode that accepts electrons; a cathode chamber housing a cathode that receives the electrons from the anode and at which the electrons moving from the anode chamber react with externally fed oxygen and water to produce hydroxide ions; and an anion exchange membrane for blocking movement of the polyvalent cations of the electrolyte into the anode chamber. In certain embodiments, the peptides of this invention can be applied to the anode, and/or cathode, and/or solution to catalyze the hydrogen producing reaction.

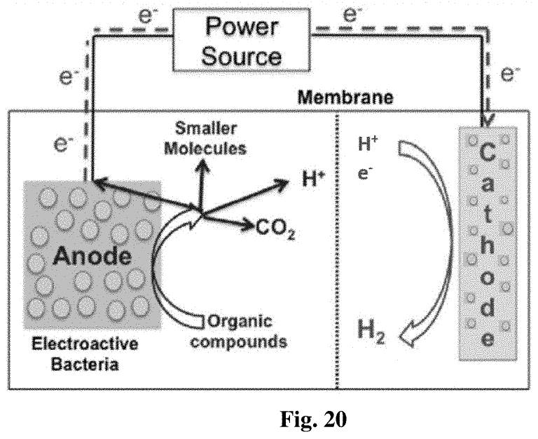

Microbial fuel cells are well known to the skilled artisan. See, e.g. C. Santoro et al. Microbial fuel cells: From fundamentals to applications, A review of J Power Sources, 356 (2017), pp. 225-224. An exemplary microbial fuel cell is provided herein as FIG. 20. Microbial fuel cells may be incorporated into a bioreactor.

The materials and methods below are provided to facilitate the practice of the present invention.

### Experiment Strategy

The catalytic metal site in natural hydrogenases is mostly coordinated by cysteine. Furthermore, studies have shown that simple hydrogenase activity can be performed with non-native proteins binding Nickel using four cysteine coordination. The [Ni—Fe] hydrogenase dinuclear site is coordinated by four cysteines separated by several hundred residues in sequence. Toward the goal of developing a minimalist peptide catalyst, we examined metal coordination sites in natural proteins where ligands were much closer together. Based on previous nickel-based hydrogen evolving catalysts, we began by designing a mononuclear nickel site.

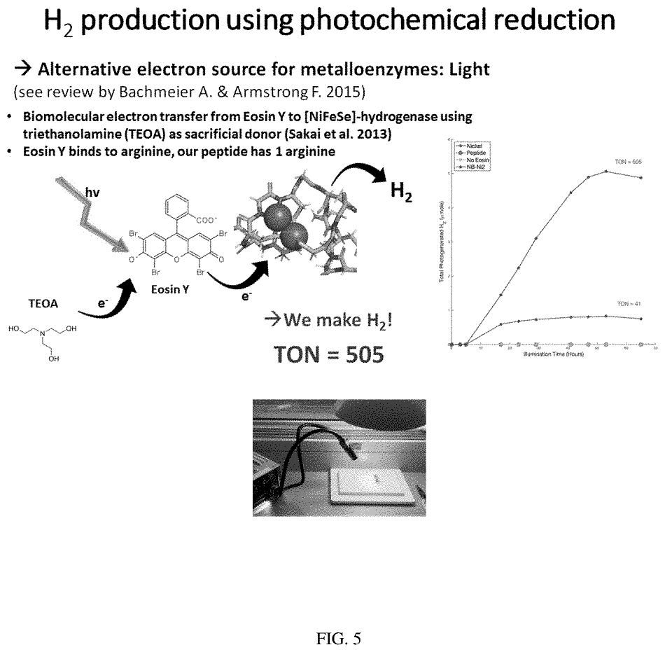

The initial scaffold was based on the high resolution structure of a Mn2+ binding cite at the C-terminus of parvalbumin. A manganese ion was octahedrally coordinated by four sidechain carboxylates, three of which were close in sequence, with the D×D×D motif adopting tight turns around the metal (FIG. 6A). A Ni2+ was docked in place of the Mn and the structure was manually mutated at 4 acidic amino acid positions to cysteines with rotamers that were in the best position to able to coordinate the Ni2+. Initial Nickel binding and hydrogen production studies were performed with the structure in (referred to as NB native) with success at binding Nickel.

In this case, the metal was fully coordinated, with an additional backbone carbonyl oxygen and water. Given that Ni2+ is frequently octahedral in proteins, we examined whether a derived site replacing carboxylates with thiols would appropriately bind nickel. Starting from the parvalbumin structure, residues 90-102, D90, 92, 94 and E102 were replaced with cysteine, setting sidechain rotamers to optimally coordinate the nickel ion. Using the protEvolver tool in the molecular modeling platform protCAD, the sequence of the remaining, non-ligand amino acids were chosen. protCAD is a molecular modeling platform that allows torsional sampling of backbone and sidechain degrees of freedom as well as sequence substitutions. Scoring is based on a force field that combines the AMBER ff14SB nonbonding terms, the Dunbrack bbdep rotamer library, and implicit solvation.protEvolver uses a novel genetic algorithm implementation to rapidly sample sequence space, and can be scaled to run in parallel on a number of compute nodes. protCAD and the protEvolver code are open source and available for download at: github.com/protcad/protCAD.

Several thousand structural models were generated. The top-ranked among these were subject to extended molecular dynamics simulations using AMBER. Models were solvated in a 8 Å water layer, using the TIP3P water model and the ff14SB force field. The system was minimized in steepest descent, followed by conjugate gradient minimization. Parameters for nickel were chosen to favor tetrahedral coordination with a S—Ni bond length of 2.27 Å. Periodic boundaries were set under constant volume and the system was thermalized from 0 to 300 K using Langevin dynamics and a collision frequency of 3 ps-1. MD simulations were run for 3.0 microseconds at 300 K. Calculations were performed with a 2 fs time-step. Structural sampling produced a distorted square-planar nickel-coordination geometry, despite the setting of tetrahedral constraints. This indicated that there were limitations to the modeling assumptions and the use of molecular mechanics to explore the metallo-peptide structure.

### Dinuclear Models and DFT Calculations

Observation of a second nickel binding necessitated additional metal ligands beyond the four cysteine thiols. In [Ni—Fe] hydrogenase, additional coordination of the iron was provided by cyanide and carbon monoxide, which were not explicitly incorporated as a part of metal reconstitution. Instead, the di-nickel site of ACS (PDB ID: 1MJG) and single nickel ions in Ni-SOD (PDB ID: 1T6U) and the [Ni—Fe] hydrogenase accessory protein, HypB, by backbone amides, led us to examine backbone amide nitrogens as potential metal ligands.

Two plausible models were examined, one where the distal nickel is coordinated by cysteines C1 and C3—the CNC model, and one where the distal nickel is coordinated by cysteines C3 and C5—the CGC model. Models were constructed and sampled with molecular dynamics as described above (metal-ligand parameter files available upon request). We followed molecular mechanics optimization with DFT geometric optimization of both models.

CGC and CNC model geometry optimization calculations were performed using the ORCA quantum chemistry package. Initial rounds of calculations were undertaken with the use of the BP86 level of theory using the Karlsruhe def2-SVP split valence polarization basis set applied to all atoms with the def2/J auxiliary basis set for Coulomb fitting, and the Karlsruhe def2-TZVP triple-zeta polarization basis set applied to the nickel atoms. The split-RI-J approximation was used in the approximation of Coulomb integrals. Upon convergence of the initial round of calculations, a second round of calculations were undertaken on the final structure of CGC. The second optimization run utilized the segmented all-electron contracted (SARC) Karlsruhe def2-TZVP valence triple-zeta polarization basis set with the fully decontracted def25 (SARC/J) auxiliary basis set (44) for all atoms excluding nickel, which instead utilized the contracted Karlsruhe basis set ZORA-def2-TZVPP valence triple-zeta with two sets of polarization functions applied to nickel and the coordinating sulfurs. The RIJCOSX approximation, which incorporates the resolution of identity approximation (RI) and the chain-of-spheres approximation (COSX) for the formation of Coulomb- and quantum mechanical exchange-matrices respectively were used as well. Scalar relativistic all-electron effects were handled by utilizing the zeroth-order regular approximation (ZORA) scalar relativistic Hamiltonian.

A large and conservative grid size was chosen (DefGrid3 as per ORCAs syntax) along with a tight self-consistent field (SCF) convergence threshold were used. Dispersion corrections were accounted for by using Grimme's DFT-D3(BJ) atom-pairwise dispersion correction with Becke Johnson damping and was employed both in the initial and second round of calculations. Aqueous solvation energies were calculated using the conductor-like polarizable continuum model (C-PCM) implemented in ORCA. A van der Waals-type cavity was employed to treat electrostatic solvation effects within the Gaussian charge scheme. The water solvent had a dielectric constant of 80.4 set within the CPCM module.

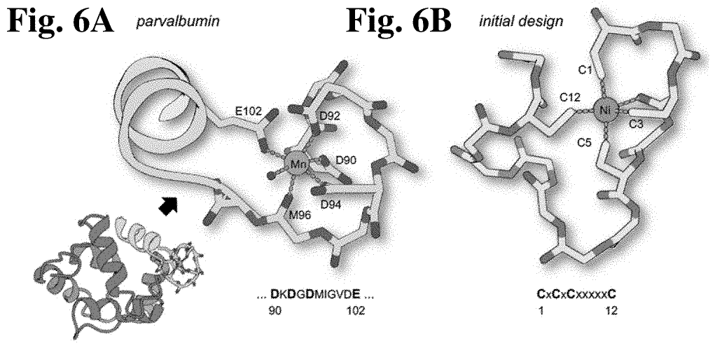

The distal nickel site in both CGC and CNC models is square planar (FIG. 7). In CGC, the proximal nickel is in the square-pyramidal coordination with 4 equatorial ligands comprising three sulfurs (C1, C5, C12) and one backbone oxygen (R11), and one axial sulfur ligand (C3). The coordination geometry of the proximal nickel in CNC is distorted square-planar which was coordinated by three sulfurs (C3, C5, C12) and one nitrogen (C12). This is inconsistent with EPR measurements, leading us to eliminate CNC model. Conversely, the coordination geometry of the proximal nickel in CGC were in concordance with EPR measurements. Although the CGC model is most consistent with EPR, we note that two α-protons are close to the proximal nickel, but were not observed by EPR, indicating further model development is still necessary.

CGC and CNC models of NB-2Ni are available in ModelArchive (modelarchive.org) under accession numbers: ma-6lwmo, ma-3g061 and ma-iyjyy.

### Peptide Synthesis

For initial peptides screening, small quantities of peptides were synthesized using a microwave-assisted peptide synthesizer (Liberty Blue, CEM Corporations) as described before (Kim et al., 2018). In brief, the synthesis was carried out using standard solid-phase methods (Roy et al 2013) with Oxyma/DIC coupling agents in DMF (Pauling & Corey, 1951) and subsequent purification with ether precipitation and RT-HPLC using a C18 column and a H₂O/acetonitrile gradient with 0.1% TFA in both solutions. The identity and purity of the peptides were confirmed using MALDI-MS analysis. Lyophilized peptide was stored at −20 C until used.

For detailed characterization, NB peptide was purchased from Genscript Biotech (Piscataway, NJ) as a lyophilized powder which was stored frozen in aliquots until used.

Peptides in this study:

NB
(SEQ ID NO: 2)
CNCGCGNNNDRCG
NB-Trp
(SEQ ID NO: 3)
CNCGCGNWNDRCG (N8W)
NBΔP
(SEQ ID NO: 13)
SNCGCGNNNDRSG (Removal of proximal site-C1S/C12S)

### NB-Nickel Reconstitution and Nickel Titrations

Lyophilized apo-NB peptides were dissolved in a buffer containing 25 mM HEPES (1-piperazineethanesulfonic acid), 100 mM NaCl and 3.75 mM of freshly prepared TCEP (Tris-2-carboxyethyl-phosphine hydrochloride), with pH adjusted to 7.5-9.2, to give a final peptide concentration of 0.75-1.0 mM. The apo-NB solution was deoxygenized by purging with 100% nitrogen gas for at least 20 min inside an airtight glass vial. Nickel reconstitution was done in an air-tight 1 cm quartz optical cuvette sealed with a septum screw cap. The cuvette was placed inside the sample compartment of a circular dichroism (CD) spectrometer (AVIV 420) with the spectrometer thermostat set at 50° C. The progress in nickel reconstitution to NB peptides was directly monitored through the CD measurements. Varying amounts of Ni²⁺ from an oxygen-free NiCl₂ stock solution were added using a purged Hamilton syringe.

For regular nickel reconstitution, the NiCl₂ concentration corresponding to 2 molar equivalents of NB peptides was added in one shot, mixed by inversion of the cuvette multiple times and incubated at 50° C. for at least 2 hours or overnight. The reconstitution at room temperature was also successful but taking substantially longer times up to 48 hours. The progress in Ni reconstitution to peptides was monitored by periodically measuring CD spectra between 300 and 600 nm, and the reconstitution was considered to be complete when the CD spectra stopped changing anymore by further incubation.

For the nickel titration experiment, small (sub-stoichiometric) aliquots of NiCl₂ were added to the peptide solutions, and the completion of each reconstitution step was monitored by CD spectrometry before addition of more NiCl₂. The titration was considered complete when further addition of NiCl₂ did not result in any changes of the CD spectra. In addition to CD experiments, circular voltammetry (CV) measurements were carried out on the same reconstitution solution at selected nickel concentration steps as described below.

### pH Titrations of Reconstituted NB-2Ni

To investigate the effect of pH on nickel coordination in NB-2Ni and on electrochemical potential, the sample buffer was modified to contain 10 mM CHES, 10 mM potassium phosphate, 10 mM MES and 10 mM HEPES, with 100 mM NaCl and 3.75 mM TCEP. The pH at each step was adjusted using NaOH/HCl, with the solution purged with nitrogen after each adjustment. CD spectroscopy was used to monitor pH-dependent changes in the NB-2Ni coordination, and circular voltammetry was used for redox titrations.

### L-Cysteine Controls

To investigate the behavior of non-specific nickel-coordination L-cysteine was reconstituted in parallel to NB peptide using equivalent conditions: 50 mM HEPES pH 7.5, 100 mM NaCl, 5 mM TCEP. 750 μM NB (containing 4 cysteines in the sequence) or 3 mM L-cysteine were dissolved in the buffer and deoxygenized by purging with 100% nitrogen gas for at least 20 min inside an airtight glass vial. 1.5 mM NiCl2 were added to each of the samples to facilitate an equivalent ratio of 4 cysteines to 2 Ni2+ in both. Reconstitutions were incubated at room temperature for 2 days (48 hours) and monitored by CD. Their electrochemical properties were investigated using circular voltammetry.

### Dynamic Light Scattering

Dynamic Light Scattering experiments were carried out on a Zetasizer (Ver 7.13, Malvern Panalytical Ltd.) in airtight, nitrogen purged quartz cuvettes using the manufacturer's standard operating protocols for size determination of ‘Standard protein’. Buffer, L-cysteine plus NiCl2 (4Cys-2Ni) and NB-2Ni were measured and analyzed using Zetasizer software v7.13.

### EPR Sample Preparation

All sample preparations were carried out in an anaerobic chamber (CoyLabs vinyl chamber, containing a nitrogen (95%)/hydrogen (5%) gas mixture).

To obtain reduced NB-2Ni complexes, 100 mL of the 2Ni-reconstituted peptides at concentration 750 mM were pre-incubated with 5-50 mM sodium bicarbonate or 5 mM K₄Fe(CN)₆ for 20 min at room temperature and then mixed with an equal volume of a buffer containing 100 mM CHES (pH 9.8) or HEPES (pH 7.5) and 50% (v/v) glycerol, before sequential addition of 1.5 mM Eu(II)Cl₂ (2 molar equivalents with respect to NB-2Ni) and 1.5 mM of diethylene-triamine-pentaacetic acid (DTPA). The reduced NB-2Ni peptides in deuterated D₂O buffers were prepared similarly, with the exception of no bicarbonate or cyanide ligands added, and using a partially deuterated d₃-glycerol. An oxidized NB-2Ni peptides were made following a similar procedure with the exception of adding a 10-50 mM Na₂IrCl₆·6H₂O oxidant during the last step.

The final concentration of the NB-2Ni peptides in all EPR samples was 375 mM. After mixing the samples were immediately transferred to 4 mm OD quartz EPR tubes and then sealed with rubber caps before taking them from the anaerobic chamber and instantly freezing in liquid nitrogen (about 30 seconds between mixing and freezing). The frozen samples were flame sealed and stored at liquid nitrogen until the experiments.

### EPR Spectroscopy

All EPR experiments were performed with a Bruker EPR spectrometer (Elexsys580e) operating at X-band microwave frequency. Helium-flow cryostats (Oxford ESR900 and CF935) equipped with an Oxford temperature controller (ITC503) were used for cryogenic temperatures.

Continuous wave (CW) EPR experiments were done at temperature 20 K for the reduced NB—Ni¹⁺ centers and at 30 K for the oxidized NB—Ni³⁺ centers. The following experimental settings were used: microwave frequency, 9.496 GHz; microwave power, 200 mW (reduced NB—Ni¹⁺) and 2 mW (oxidized NB—Ni³⁺); modulation amplitude, 1-2 mT. Concentration yields of the reduced NB—Ni¹⁺ or oxidized NB—Ni³⁺ centers in each sample were determined by comparing the integrated intensities of the measured Ni^{1+/3+} signals against the EPR standard with a known number of spins (a CuSO₄·5H₂O crystal of known weight in a mineral oil).

Pulsed EPR experiments, including two-pulse ESEEM (π/2-τ-π-τ-echo), three-pulse ESEEM (π/2-τ-π/2-T-π/2-τ-echo), four-pulse HYSCORE (π/2-τ-π/2-t₁-π-t₂-π/2-τ-echo) and Davies ENDOR (see below), were performed to characterize nuclear spin environment of reduced/oxidized Ni^{1+/3+} centers. Phase cycling was used as required in each pulsed experiment to eliminate contributions from unwanted echoes (Schweiger A., Jeschke G., Principles of Pulse Electron Paramagnetic Resonance. Oxford University Press: 2001).

Prior to Fourier transformation (FT), the ESEEM/HYSCORE time-domains were baseline corrected by fitting the oscillating decays with stretched exponential decay functions, then dividing the experimental decay by the fit function and finally subtracting a unity. This baseline correction procedure resulted in FT ESEEM spectral intensities which were automatically normalized to a unit echo signal amplitude. This normalization procedure allowed us to directly compare spectral amplitudes in ESEEM spectra measured for different samples, with different Ni^{1+/3+} concentrations, etc. See Dikanov S. A., Tsvetkov Yu. D., Electron Spin Echo Envelope Modulation (ESEEM) Spectroscopy, CRC Press (1992)

After Fourier transformation, linear phase correction was applied to the FT spectra in order to correct for missing dead times. In case of two-pulse ESEEM, the dead time (t₀) was determined by initial t delay between the π/2 and π pulses in the experiment. In case of three-pulse ESEEM, the correction time was calculated as (t+t₀), where t is the fixed delay between the first π/2 and second π/2 pulses in the three-pulse sequence, and t₀ is the initial delay between the second and third pulses. In case of HYSCORE, the linear phase correction was applied in both directions with the correction time calculated as (t/2+t₀), where t₀ is the initial delay between the second and third pulses, or the third and fourth pulses. All ESEEM/HYSCORE spectra presented in this work are shown as normalized and phase-corrected cosine Fourier-Transforms.

Other experimental pulsed EPR settings: microwave frequency, 9.776 GHz; magnetic fields, 309 mT (the g_{∥} field orientation) and 336 mT (the g_{\hat{ }} field orientation) for reduced NB—Ni¹⁺ centers, and 345 mT (g_{∥}) and 309-318 mT (g_{\hat{ }}) for oxidized NB—Ni³⁺ centers; microwave p/2 and p pulses, 16 and 32 ns, respectively; initial t delay, 104 ns (2-pulse ESEEM) and 40 ns (3-pulse ESEEM/HYSCORE); integration window, 16 ns (ESEEM/HYSCORE) and 60 ns (field-sweep echo-detected EPR); shot repetition times, 1-2 ms; and temperature, 20-30 K. All EPR/ESEEM simulations were performed using the EasySpin toolbox for MATLAB (available on the world wide web at easyspin.org/).

### Electrochemical Measurements

All electrochemical experiments were carried out in an anaerobic atmosphere continuously purging nitrogen gas though the solution and headspace of the electrochemical cell. The cyclic voltammetry experiments were set up using a Bio-logic potentiostat (EC50) and a three-electrode configuration with an Ag/AgCl electrode (BASi) as a reference and a platinum wire as a counter electrode. A glassy carbon electrode (company) was used as a working electrode and it was polished with (1 mM) alumina slurry prior to every measurement. For every experiment, 5 mL of NB—Ni peptide at various concentrations were filled into a 3-port Echem cell and connected to the electrodes while purging the solution with N₂. During the CV scan the purging needle was withdrawn from the solution while still purging the headspace. Data were acquired using the EC-lab software (V10.44) and then baseline corrected and analyzed using MATLAB. All potentials quoted in this work were referenced to a standard hydrogen electrode (SHE) and calculated as E(SHE)=E(Ag/AgCl)+205 mV.

### Photochemical Experiments

Photocatalytic activity of Ni-reconstituted NB peptides toward H₂ evolution was examined in presence of Eosin Y dye (−910 mV vs SHE) as a photosensitizer and triethanolamine (TEOA) as a sacrificial electron donor (11). The samples (10 μM NB, 500 μM Eosin Y, 200-500 mM TEOA in a HEPES buffer, pH 8.0 with 100 mM NaCl) of typical volume 1 ml were placed in air-tight 13.5 ml glass vials sealed with 10 mm-thick rubber stopper. Before illumination the samples were purged with nitrogen gas for at least 20 minutes to remove any traces of oxygen.

The samples were placed into the box lined with aluminum foil from inside and were illuminated with green light (540 nm) from two 5 W LED bulbs installed in the microscope light source. The incident light intensity on the samples ˜0.2 mW/cm2 was measured using an Ophir photodiode power meter (Nova II with PD300-3W-V1). During the course of the photochemical reaction the amount of H₂ gas produced in the headspace of the vials was probed periodically using a gas chromatographer (SRI Instruments, Model 310) equipped with a TDC detector and using nitrogen as a carrier gas.

### H₂ Calculation

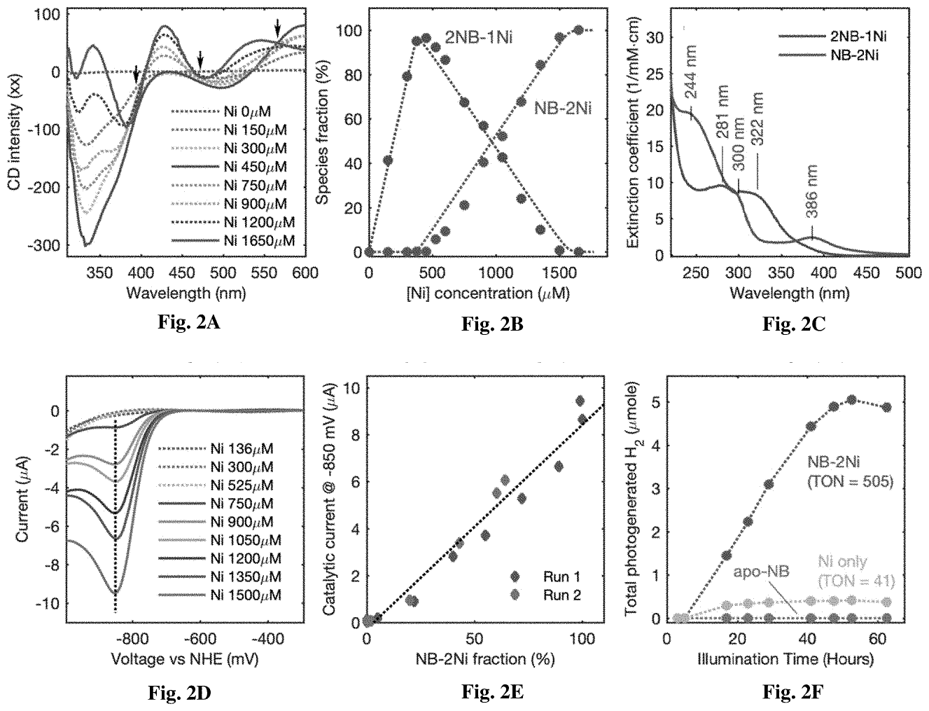

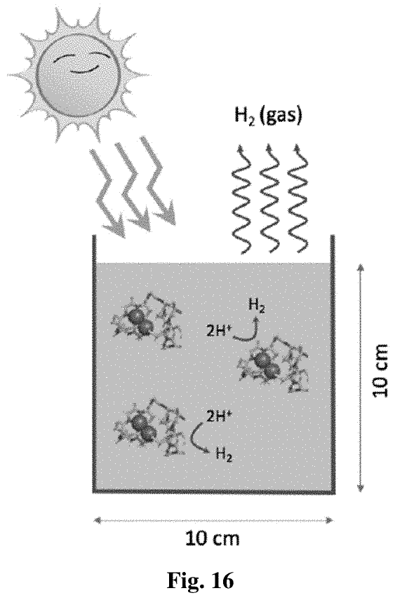

Our model considers an air-open water container with dimensions of 10×10×10 cm³ (total volume 1 liter) and NB-2Ni peptide concentration C_{NB} as illustrated in FIG. 17. Under sun illumination, the NB-2Ni peptides start producing H₂ uniformly through the volume of the container at a constant rate Rₚₕₒₜₒ=0.2 H2/min as measured in our photochemical experiments (FIG. 2F). This photochemically generated H₂ can then diffuse to the top surface of the container where it can irreversibly escape from solution to atmosphere. At the beginning of the reaction, when the H2 concentration in solution is low, the escape rate is also low. However, as the photochemical reaction proceeds and higher H₂ concentrations accumulate, the escape rate also increases proportionally, eventually reaching a steady-state equilibrium when the rate of overall H₂ production by all NB's in the container is compensated by the rate of H₂ escape from the container to the atmosphere:

[H₂]_{steady-state}·vₚ·S_{surf}=Rₚₕₒₜₒ·C_{NB}·V.

Here, [H₂]steady-state is the steady-state H₂ concentration in the container, C_{NB} is the NB-2Ni concentration, Rₚₕₒₜₒ=0.2 H₂/min is the H₂ photocatalytic rate of H₂ production (as measured from our photochemical experiments, FIG. 2F), V is the container volume and S_{surf} is its top surface area exposed to the atmosphere, and vₚ=1.3*10-2 cm/sec is the so-called piston velocity that controls the diffusion rate of H₂ from solution to atmosphere in an open container.

By assuming the NB-2Ni concentration of C_{NB}=10 nM in solution, we can immediately estimate that the steady-state concentration will reach [H₂]_{steady-state}=26 nM. This [H₂] is 5 times larger than required for active methanogenesis. It is also much higher than the average 1 nM concentration of dissolved H₂ in contemporary oceans.

The following example is provided to illustrate certain embodiments of the invention. It is not intended to limit the invention in any way.

### Example I

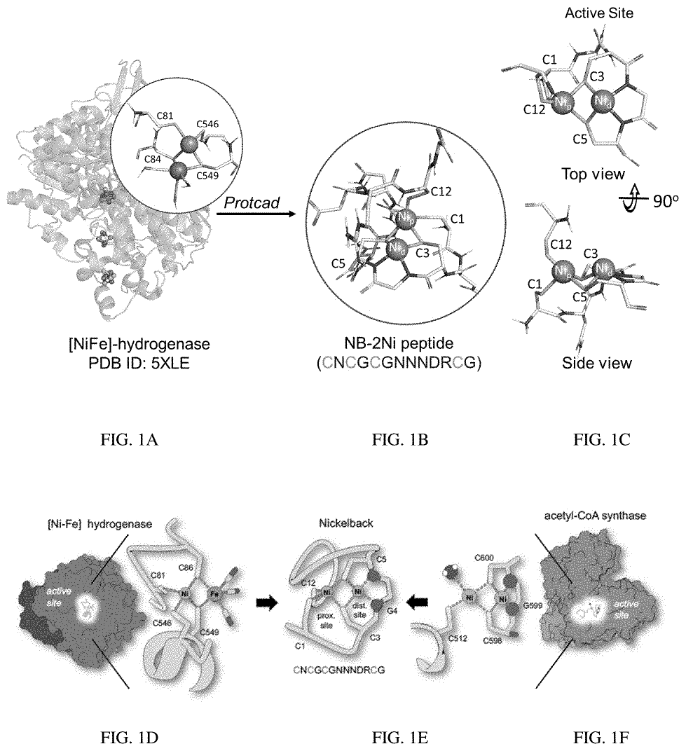

Currently, molecular hydrogen (H₂) is a source of energy for only specialized microorganisms in anaerobic environments. However, early in Earth's history the first microbial metabolisms were dependent on the H₂ gas (1, 2). Reversible biological oxidation of H₂ is catalyzed by hydrogenases, redox metalloenzymes with iron-iron (FeFe), nickel-iron (NiFe), or iron-only (Fe) active sites (3, 4). Phylogenetic studies suggest that [NiFe]-hydrogenases are the most ancestral; soluble nickel and iron ions were far more abundant in the Archean and Proterozoic oceans than today (4). These hydrogenases are complex nanomachines (FIG. 1) that comprise chains of several hundreds of amino acids, multiple subunits, and multiple metal cofactors that ferry electrons to the active Ni—Fe site (3). However, the ancestral hydrogenase must have been much smaller and simpler. Model studies on a Ni-substituted rubredoxin show that a simple fold ˜50 amino acids can evolve H₂ (5).

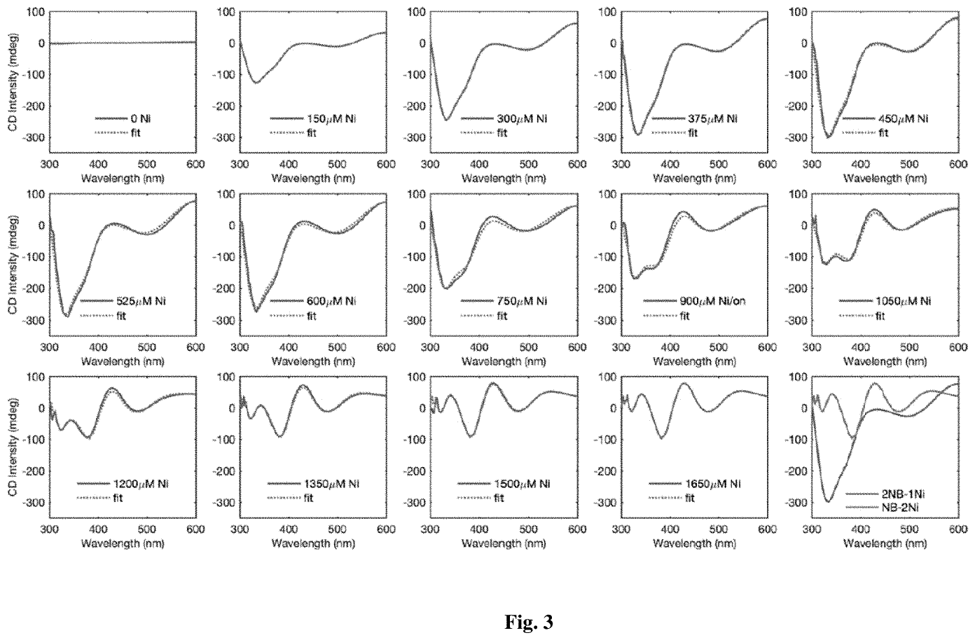

In the extant [NiFe]-hydrogenase the active site Ni—Fe ions are coordinated by four cysteines and several small molecules, like CN⁻, CO and water (FIG. 1A). However, the cysteines are separated by hundreds of amino acids implying a long sequence that is too complex for a minimal design analogous to the natural enzyme. To achieve a compact design, we started with the CGC motif known to coordinate di-nickel clusters in acetyl-CoA synthase (ACS, pdb: 1RU3). The CGC provides two backbone amide and two sidechain thiol ligands in a planar-square geometry to coordinate one (distal) Ni²⁺ ion, while the same thiol ligands also serve as bridging ligands to coordinate the second (proximal) Ni²⁺ ion (FIG. 3). We extended the sequence by adding two extra cysteines, CXCGC(X)ₙCG, to serve as auxiliary ligands for the second proximal Ni²⁺. Using further analysis naturally occurring minimal metal binding motifs (FIG. 6), the scaffold was adjusted to follow the pattern: CXCGCXXXXXCG (SEQ ID NO: 12).

The positions for these two cysteines were chosen based on the minimal metal binding motifs reported in (Gutter, 2015). Remaining variable positions (X's) were selected computationally using protCAD (6) resulting in the Nickel Back (NB) class of designs (See Experimental Strategy). Three possible di-nickel binding configurations within the NB peptide were envisioned (FIG. 3), with only one configuration, shown in FIG. 1B, consistent with our spectroscopic data. In the final design, metal coordination mimicked features of the ACS di-nickel binding site (FIG. 1F). As in ACS, a glycine (g4) and cysteine (C5) provide two backbone amindes. Two sidechain thiols (C3, C5) complete the (distal) Ni²⁺ ion coordination with a square-planar geometry. C3 and C5 also serve as bridging ligands to a second (proximal) Ni²⁺ ion whose coordination is completed with the remaining cysteines C1 and C12. We define proximal/distal sites based on nomenclature from equivalent positions of nickels in ACS and hydrogenase. Coordinates for the DFT optimized model of NB-2Ni are provided on ModelArchive (FIG. 7).

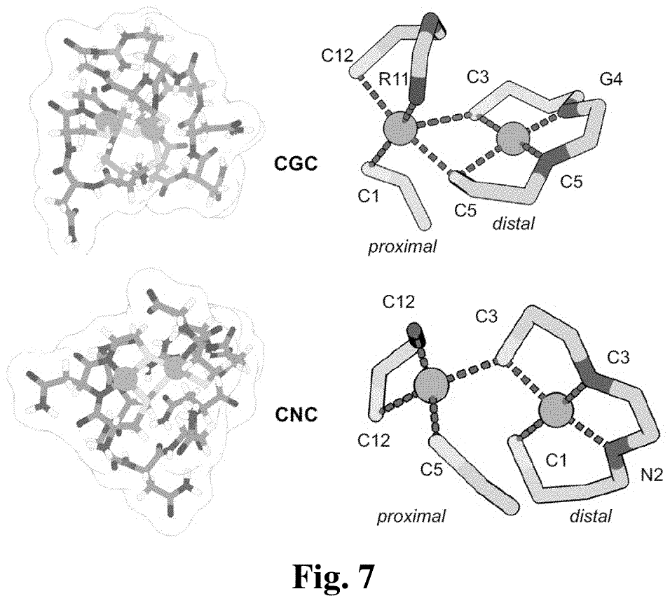

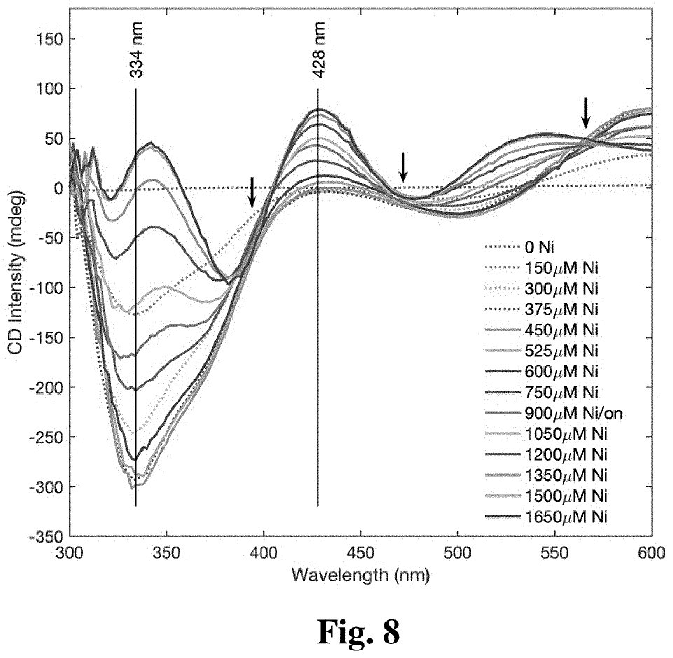

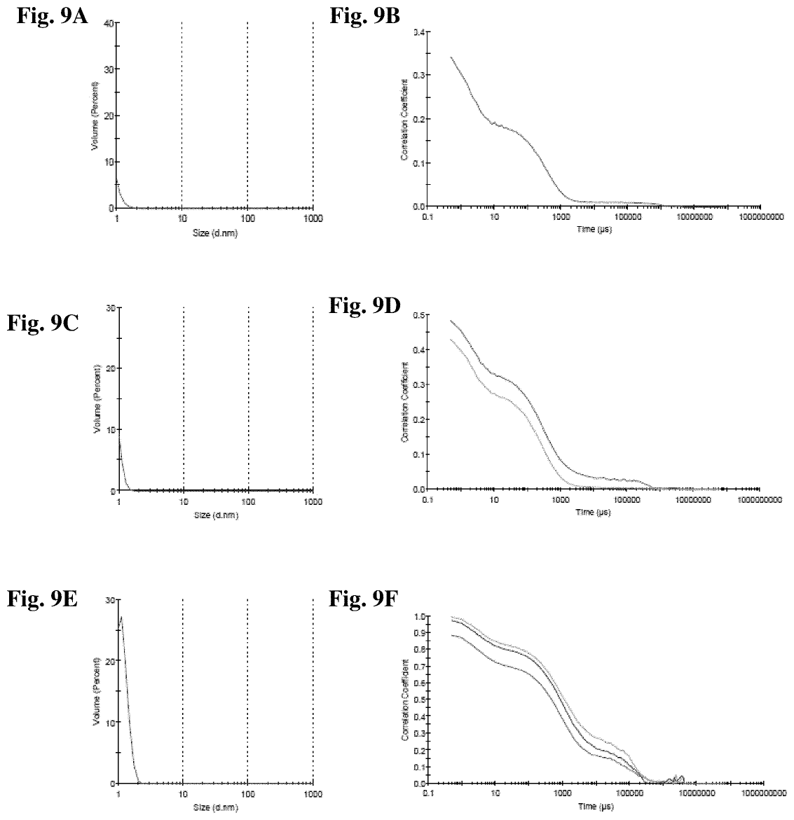

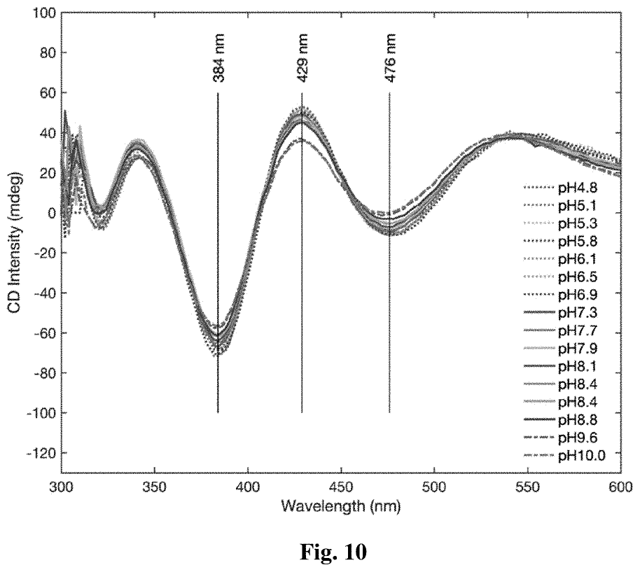

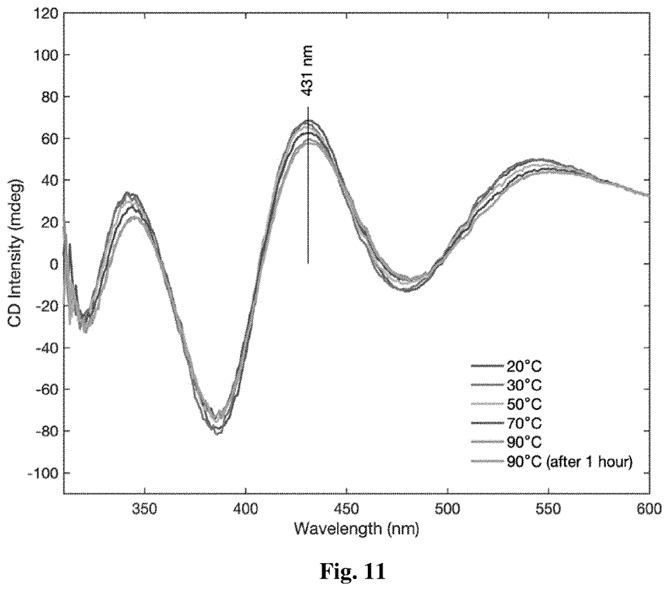

Apo-NB was produced by Fmoc solid-phase synthesis (7) and reconstituted with Ni²⁺ salts at 50° C., while monitoring the progress by UV-visible absorption and circular dichroism (CD) spectroscopy (FIG. 2A,C). Two optically active species were identified: an assembly intermediate (2NB-1Ni) with the 2:1 (peptide:Ni) stoichiometry, and the final assembly (NB-2Ni) saturating at the 1:2 stoichiometry. The isosbestic points in the CD spectra (marked with arrows in FIG. 2A) indicated a direct transformation from 2NB-1Ni to NB-2Ni during the reconstitution. Spectral decomposition of the CD spectra using the two component model (FIGS. 8 and 3) let us to determine the fractional concentrations of 2NB-1Ni and NB-2Ni in the course of Ni reconstitution (FIG. 2B). Dynamic light scattering (DLS) measurements of NB-2Ni showed a dominant monodisperse species with a radius of hydration consistent with a compact monomeric peptide (FIG. 9). The fully reconstituted NB-2Ni was confirmed to be stable at pH 5.5-10, at temperatures from 20 to 90° C., and even in the presence of molecular oxygen (FIGS. 10-12), suggesting NB would have been stable over a range of predicted ocean pH and temperatures during the Archean eon.

The fully reconstituted NB-2Ni was confirmed to be stable in a broad pH range from 5.5 to 10 and at temperatures from 20 to 90° C. (FIG. 3).

In parallel with optical studies, we probed the redox activity of both the intermediate 2NB-1Ni and the mature NB-2Ni complexes using cyclic voltammetry (FIG. 2D, 13-14). The catalytic current at −850 mV (vs SHE, standard hydrogen electrode) clearly correlated with the increasing NB-2Ni fraction (FIG. 2E), confirming its electrochemical activity. This redox potential is more than sufficient to drive hydrogen evolution.

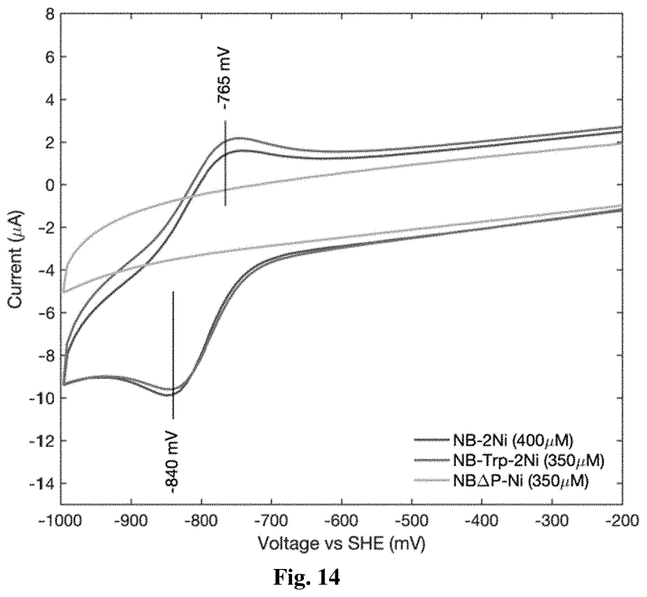

Control studies on free cysteine complexed with metal showed that while nickel chelates are formed, they have a very different coordination structure compared to NB-2Ni as measured by CD (FIG. 15). Cysteine-Ni chelates did not form monodisperse species as measured by DLS (FIG. 9). Cyclic voltammetry confirmed that cysteine-Ni chelates did not exhibit a catalytic current consistent with reductive evolution of hydrogen. Together, these observations are consistent with NB-2Ni forming a stable, unique coordination complex capable of redox chemistry, that cannot be attributed to nonspecific metal-thiol complexes.

To establish that NB-2Ni could catalytically evolve hydrogen, we used a photochemical assay with the organic dye (Eosin Y, −910 mV vs SHE) as a photosensitizer, triethanolamine (TEOA) as sacrificial electron donors, and green 540 nm LED light (8). H₂ evolution was quantified using gas chromatography. At pH 8, the turnover number TON=500 and the turnover frequency TOF=0.2 H₂/min were observed (FIG. 2F). This activity is on par with TOF=0.1-0.9 H₂/min reported for other Ni-substituted proteins (9, 10), however our TON number is substantially higher.

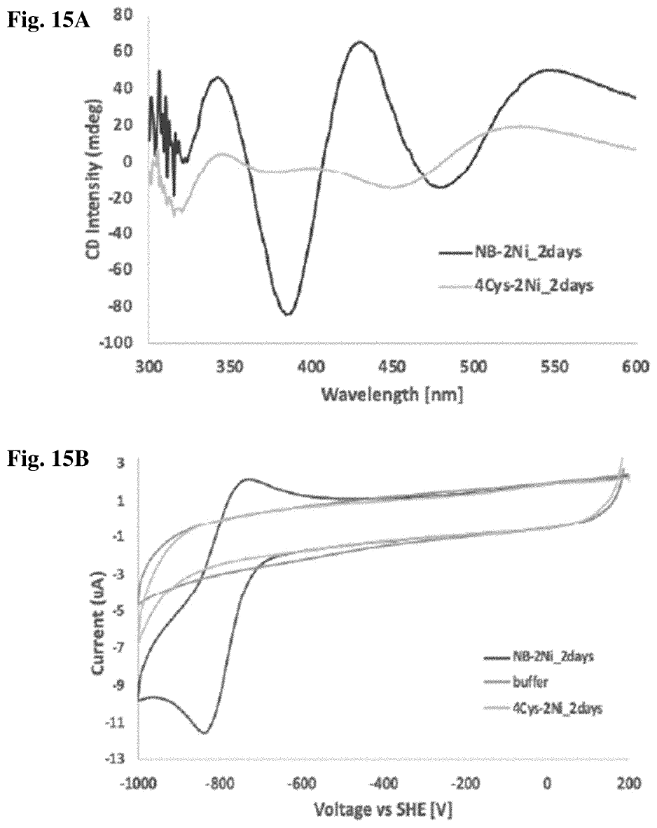

Several of the early emerging metabolisms that include sulfate reduction, nitrate reduction, iron reduction and methanogenesis operate at minimum available energy (i.e., ˜<−300 kJ/per reaction under standard condition) (1, 11-13). Most of this available energy is lost as heat or other processes (e.g., proton pump) so only ˜30-40% of that energy goes to ATP production and growth (11). While these processes rely heavily on donor concentration but because of low energy differences even a nano-Molar (nM) range of electron donor concentration can make these processes thermodynamically favorable. For example, active iron (Fe) reducers in marine sediments have been observed at ˜1 nM of [H₂] (11, 12). Our peptide can maintain a steady [H₂] concentration of >20 nM (FIG. 16 and Methods discussed above). This is more than enough to support sulfate reducers, nitrate reducers, iron reducers and methanogens (11, 12, 14). In addition, ancestral organisms likely did not also have sophisticated additional metabolisms as these modern anerobic organisms have suggesting that our peptide is more than capable of supporting ancestral life on early Earth.

We attempted to recreate ancestral [NiFe]-hydrogenases that likely were sluggish and slow. It is a valid assumption given the competition and demand for a better catalyst was very low on early Earth. It is the evolution of biosphere and geosphere (e.g., emergence of oxygen) that injected more entropy in the system creating opportunity for new metabolisms to emerge. This created competition, toxicity (e.g., radical oxygen) amongst microbial population which forced these proteins to evolve both in efficiency and specificity.

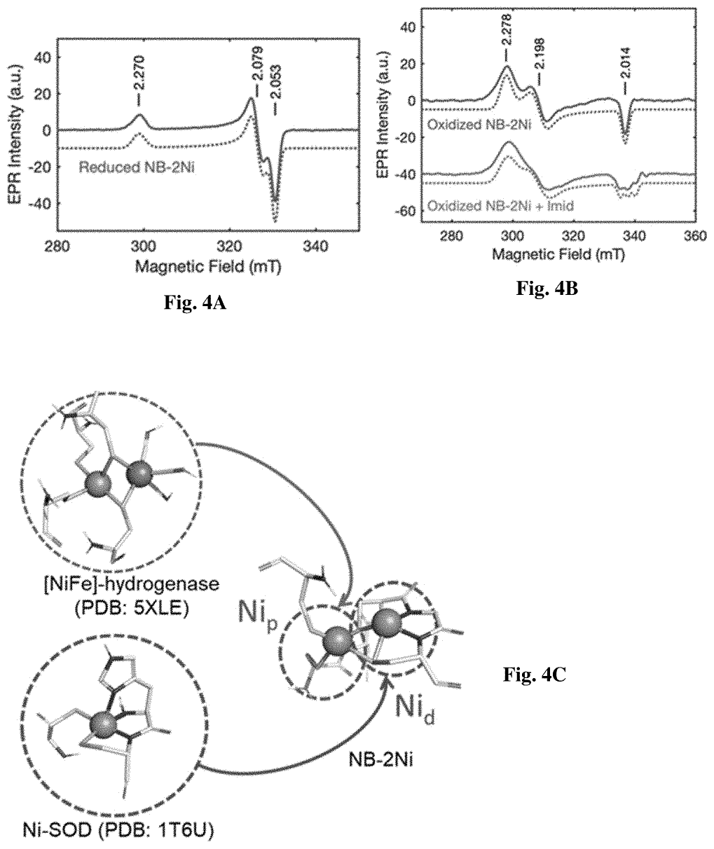

The di-nickel nature of NB-2Ni was confirmed in our EPR experiments. The resting state of NB-2Ni is not EPR active, as both nickels are in the 2+ oxidation state. However, it was possible to trap one-electron reduced NB-2Ni in the presence of Eu(II)DTPA (14), and a small ligand, either CN- or bicarbonate. In case of reduced NB-2Ni (FIG. 4A), the anisotropic EPR signal with g_{∥}>g_{\hat{ }} was observed characteristic of a d⁹ electron configuration, with Ni¹⁺ in distorted octahedral or square planar environment, and with the unpaired electron preferentially residing on a d_{x2-y2} orbital (15, 16). Both the proximal and distal Ni sites in NB-2Ni have this type of coordination symmetry (FIG. 1), and thus both sites should be considered as candidates.

The EPR spectra of reduced NB-2Ni (FIG. 4A and FIG. 3), as well as the ESEEM and Hyscore spectra, showed no evidence of strongly interacting ¹⁴N nitrogens that would be expected for Ni¹⁺ located at the distal Ni(S₂N₂) site, where its d_{x2-y2} orbital would strongly overlap with two equatorial amide ¹⁴N nitrogens, leading us to exclude the distal Ni(S₂N₂), and assign the observed EPR signal to the proximal Ni¹⁺ site with S-only coordination, e.g. Ni¹⁺(S₄). EPR signals of similar symmetry with g_{∥}>g_{\hat{ }} have been reported for the reduced Ni¹⁺ state (the “Ni-L” state) of [NiFe]-hydrogenase (17) and also for the proximal Ni¹⁺ site of acetyl-CoA synthase (18). In both cases Ni¹⁺ ions were found in the distorted octahedral environments being coordinated by 3-4 thiol ligands and no nitrogen ligands, e.g. Ni(S₃₋₄Xₙ) (FIG. 4A). Three-pulse ESEEM spectra and 2D HYSCORE cross-peaks of the reduced NB-2Ni are consistent with a nearby nitrogen and exchangeable proton that disappears in D2O. These are tentatively assigned to nearby backbone amide hydrogen bonds to proximal nickel coordinating cysteine sulfurs, similar to those observed in previous metalloprotein designs.

The EPR signal of opposite symmetry, with g_{\hat{ }}>g_{∥}, was observed in case of oxidized NB-2Ni (FIG. 4B). The signal was characteristic of Ni³⁺ in the d⁷ electron configuration with an unpaired electron residing on a d_{z2} orbital (15, 16). Square-planar or elongated octahedral Ni³⁺ coordination would fit the observed g-factor symmetry. Remarkably, both the shape and the g-factor values of the oxidized NB-2Ni signal were similar to those reported for oxidized nickel superoxide dismutase (Ni-SOD, pdb: 1T6U), where Ni³⁺ is in a square-pyramidal coordination with two backbone amide nitrogens and two cysteine thiolates in a square-planar Ni(S₂N₂) geometry with an additional histidine nitrogen coordinated in the axial position (19). The spectral resemblance to Ni-SOD improved even further when we added 10 mM imidazole to the NB-2Ni sample (FIG. 4B—red trace), with the ¹⁴N hyperfine splitting now resolved on the g_{∥}=2.014 peak indicating an axial imidazole coordination similar to Ni-SOD (19).

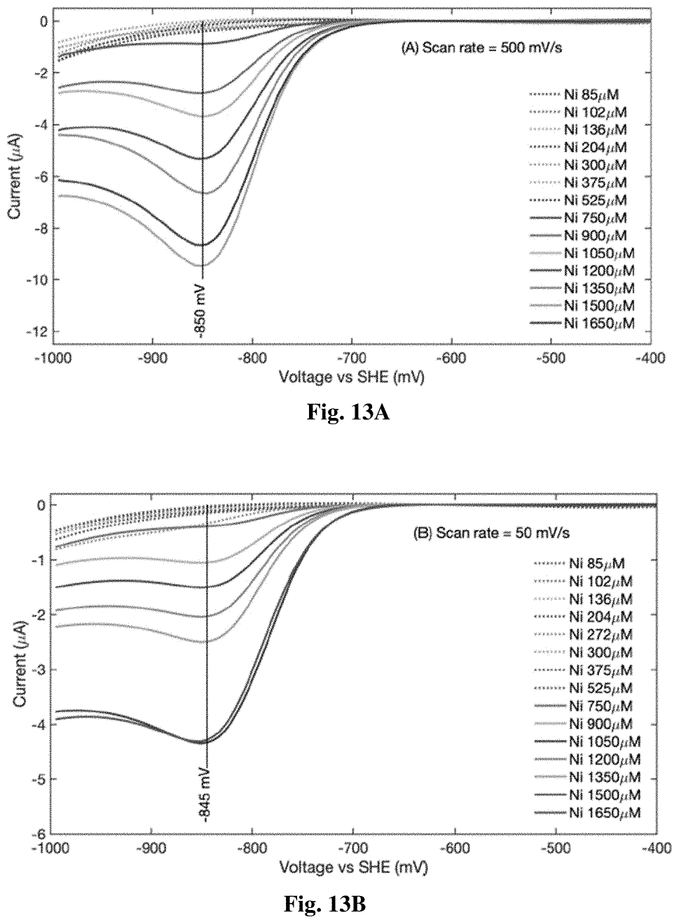

Based on this comparison, the EPR signal in oxidized NB-2Ni was attributed to the distal Ni³⁺(S₂N₂) site. Importantly, the square-planar S₂N₂ configuration of this distal Ni³⁺ was distinctly different from the octahedral S₄Xₙ coordination found for the proximal Ni¹⁺ site in reduced NB-2Ni. Together, our analysis supports a NB-2Ni with two distinct nickel sites resembling the ACS-like di-nickel cluster featuring a proximal Ni, which is easier to reduce, and a distal Ni, that is easier to oxidize. Further, we would expect removing C1 and C12 might eliminate the proximal site while retaining the distal one. We designed NBDP with C1 and C12 replaced by serine. Nickel titrations of NBDP saturated at a 1:1 complex, with a distinct CD spectrum compared to NB-2Ni (FIG. 17). EPR of oxidized NBDP-Ni is comparable with oxidized NB-2Ni, confirming the assignment of Ni3+ at the distal site. No catalytic wave was observed for NBDP-Ni in cyclic voltammetry (FIG. 14), indicating the distal Ni alone is insufficient for catalysis.

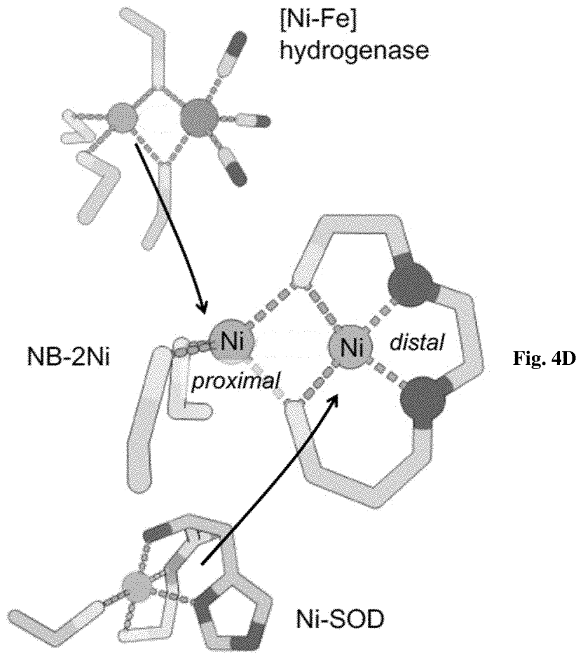

FIG. 5 shows the hydrogen production using photochemical reduction.

### Discussion

The kinetic catalytic parameters of NB-2Ni are sufficient to function as the electron sink in a metabolic half-cell, where evolution and diffusion of hydrogen gas could maintain a redox disequilibrium. Assuming a peptide concentration of 10 nM, just a few molecules/cell, lower than hydrogenase abundances in modern organisms, NB-2Ni could maintain a steady H₂ concentration of >20 nM (See FIG. 17 and Methods). For comparison, active iron reducers in marine sediments have been observed at ˜1 nM of [H₂]. An NB-like peptide could have plausibly served as an electron sink in early metabolic pathways. Notably, we have not observed hydrogen evolution in this system, likely due to the overpotential required for catalysis. To drive metabolisms such as methanogenesis/acetogenesis, H₂-oxidizing catalysts would have needed to be present.

Many structural and chemical analogies exist between NB and ancient enzymes such as ACS and [Ni—Fe] hydrogenase. However, intermediates in the evolution from peptides to single- and multi-domain oxidoreductases are too ancient to reconstruct by phylogenetic methods. However, clues to the evolution of these sites rise from protein interaction networks. The HypB protein responsible for transferring nickel to [Ni—Fe] hydrogenase in E. coli shares structural features with NB—notably a CGC motif that coordinates nickel through a backbone amide. The evolution of protein interaction networks by duplication and diversification can be used to reconstruct intermediates in this process.

Simple metal-binding peptides were likely an essential step in the origins of life. However, engineering candidate peptide catalysts to test this idea led us to the surprising observation that short sequences can exhibit significant stability and activity. This challenges a paradigm that early enzymes were poor catalysts and that molecular evolution has optimized their stability and activity. Similarly, the designed 12-amino acid protein, ambidoxin, complexed a 4Fe-4S cluster with four cysteines, and was capable of hundreds of reversible, redox cycles (19). Ambidoxin was designed to mimic the active site of bacterial ferredoxins, natural protein electron carriers which were five-fold larger.

These two successes point to a general approach for engineering small peptide catalysts by structural analysis of large metalloenzyme active sites. The chemical stability and functional potential of small peptides complexed with transition metals make them plausible ancestors in the evolution of oxidoreductases.

### REFERENCES

### References

1. P. G. Falkowski, T. Fenchel, E. F. Delong, The Microbial Engines That Drive Earth's Biogeochemical Cycles. Science (80-.). 320, 1034-1039 (2008).
2. K. H. Nealson, F. Inagaki, K. Takai, Hydrogen-driven subsurface lithoautotrophic microbial ecosystems (SLiMEs): do they exist and why should we care? Trends Microbiol. 13, 405-410 (2005).
3. W. Lubitz, H. Ogata, O. Rüdiger, E. Reijerse, Hydrogenases. Chem. Rev. 114, 4081-4148 (2014).
4. J. W. Peters, G. J. Schut, E. S. Boyd, D. W. Mulder, E. M. Shepard, J. B. Broderick, P. W. King, M. W. W. Adams, [FeFe]- and [NiFe]-hydrogenase diversity, mechanism, and maturation. Biochim. Biophys. Acta—Mol. Cell Res. 1853, 1350-1369 (2015).
5. J. Slater, W., S. C. Marguet, H. A. Monaco, H. S. Shafaat, Going beyond Structure: Nickel-Substituted Rubredoxin as a Mechanistic Ml. J. W. Slater, S. C. Marguet, H. A. Monaco, H. S. Shafaat, Going beyond Structure: Nickel-Substituted Rubredoxin as a Mechanistic Model for the [NiFe] Hydrogenases. J. Am. Chem. Soc. J. Am. Chem. Soc. 140, 10250-10262 (2018).
6. D. H. Pike, V. Nanda, Empirical Estimation of Local Dielectric Constants: Toward Atomistic Design of Collagen Mimetic Peptides. Biopolymers. 104, 360 (2015).
7. R. B. Merrifield, Solid Phase Peptide Synthesis. I. The Synthesis of a Tetrapeptide. J. Am. Chem. Soc. 85, 2149-2154 (2002).
8. A. Bachmeier, F. Armstrong, Solar-driven proton and carbon dioxide reduction to fuels—lessons from metalloenzymes. Curr. Opin. Chem. Biol. 25, 141-151 (2015).
9. M. J. Stevenson, S. C. Marguet, C. R. Schneider, H. S. Shafaat, Light-Driven Hydrogen Evolution by Nickel-Substituted Rubredoxin. ChemSusChem. 10, 4424-4429 (2017).
10. D. Selvan, P. Prasad, E. R. Farquhar, Y. Shi, S. Crane, Y. Zhang, S. Chakraborty, Redesign of a Copper Storage Protein into an Artificial Hydrogenase. ACS Catal. 9, 5847-5859 (2019).
11. D. E. Canfield, E. Kristensen, B. Thamdrup, Thermodynamics and Microbial Metabolism. Adv. Mar. Biol. 48, 65-94 (2005).
12. T. M. Hoehler, M. J. Alperin, D. B. Albert, C. S. Martens, Thermodynamic control on hydrogen concentrations in anoxic sediments. Geochim. Cosmochim. Acta. 62, 1745-1756 (1998).
13. E. K. Moore, B. I. Jelen, D. Giovannelli, H. Raanan, P. G. Falkowski, Metal availability and the expanding network of microbial metabolisms in the Archaean eon. Nat. Geosci. 2017 109. 10, 629-636 (2017).
14. D. E. Canfield, M. T. Rosing, C. Bjerrum, Early anaerobic metabolisms. Philos. Trans. R. Soc. B Biol. Sci. 361, 1819 (2006).
15. M. V. Gastel, W. Lubitz, EPR Investigation of [NiFe] Hydrogenases. J. Am. Chem. Soc., 441-470 (2009).
16. A. Abragam, B. (Brebis) Bleaney, Electron paramagnetic resonance of transition ions, 277-345 (2012).
17. M. E. Pandelia, H. Ogata, L. J. Currell, M. Flores, W. Lubitz, Inhibition of the [NiFe] hydrogenase from Desulfovibrio vulgaris Miyazaki F by carbon monoxide: An FTIR and EPR spectroscopic study. Biochim. Biophys. Acta—Bioenerg. 1797, 304-313 (2010).
18. G. Bender, T. A. Stich, L. Yan, R. D. Britt, S. P. Cramer, S. W. Ragsdale, Infrared and EPR Spectroscopic Characterization of a Ni(I) Species Formed by Photolysis of a Catalytically Competent Ni(I)—CO Intermediate in the Acetyl-CoA Synthase Reaction. Biochemistry. 49, 7516-7523 (2010).
19. David P. Barondeau, Carey J. Kassmann, ‡Cami K. Bruns, and John A. Tainer, E. D. Getzoff*, Nickel Superoxide Dismutase Structure and Mechanism†. Biochemistry. 43, 8038-8047 (2004).
20. R. Hidalgo, P. A. Ash, A. J. Healy, K. A. Vincent, Infrared Spectroscopy During Electrocatalytic Turnover Reveals the Ni-L Active Site State During H2 Oxidation by a NiFe Hydrogenase. Angew. Chemie Int. Ed. 54, 7110-7113 (2015).
21. E. Smith, H. J. Morowitz, Universality in intermediary metabolism. Proc. Natl. Acad. Sci. 101, 13168-13173 (2004).
22. H. Liu, K. O. Konhauser, L. J. Robbins, W.-d. Sun, Global continental volcanism controlled the evolution of the oceanic nickel reservoir. Earth and Planetary Science Letters 572, 117116 (2021).
23. R. J. P. Williams, The bakerian lecture, 1981 natural selection of the chemical elements. Proceedings of the Royal Society of London. Series B. Biological Sciences 213, 361-397 (1981).
24. O. Gutten, L. Rulíšek, How simple is too simple? Computational perspective on importance of second-shell environment for metal-ion selectivity. Physical Chemistry Chemical Physics 17, 14393-14404 (2015).
25. J. Krissansen-Totton, G. N. Arney, D. C. Catling, Constraining the climate and ocean pH of the early Earth with a geological carbon cycle model. Proceedings of the National Academy of Sciences 115, 4105-4110 (2018).
26. K. A. Vincent et al., Instantaneous, stoichiometric generation of powerfully reducing states of protein active sites using Eu (II) and polyaminocarboxylate ligands. Chemical communications, 2590-2591 (2003).
27. J. D. Kim et al., Minimal heterochiral de novo designed 4Fe-4S binding peptide capable of robust electron transfer. Journal of the American Chemical Society 140, 11210-11213 (2018).
28. V. Nanda et al., De novo design of a redox-active minimal rubredoxin mimic. Journal of the American Chemical Society 127, 5804-5805 (2005).
29. K. Gutekunst et al., In-vivo turnover frequency of the cyanobacterial NiFe-hydrogenase during photohydrogen production outperforms in-vitro systems. Scientific reports 8, 1-10 (2018).
30. H. Raanan, S. Poudel, D. H. Pike, V. Nanda, P. G. Falkowski, Small protein folds at the root of an ancient metabolic network. Proceedings of the National Academy of Sciences 117, 7193-7199 (2020).
31. K. C. Chan Chung et al., A high-affinity metal-binding peptide from Escherichia coli HypB. Journal of the American Chemical Society 130, 14056-14057 (2008).
32. N. H. Horowitz, On the evolution of biochemical syntheses. Proceedings of the National Academy of Sciences 31, 153-157 (1945).
33. D. Rossetto, S. S. Mansy, Metals Are Integral to Life as We Know It. Frontiers in Cell and Developmental Biology 10, (2022).
34. F. Baymann et al., The redox protein construction kit: pre-last universal common ancestor evolution of energy-conserving enzymes. Philosophical Transactions of the Royal Society of London. Series B: Biological Sciences 358, 267-274 (2003).
35. J.-P. Declercq, B. Tinant, J. Parello, J. Rambaud, Ionic interactions with parvalbumins: crystal structure determination of pike 4.10 parvalbumin in four different ionic environments. Journal of molecular biology 220, 1017-1039 (1991).
36. L. r. Rulíšek, J. Vondrášek, Coordination geometries of selected transition metal ions (Co2+, Ni2+, Cu2+, Zn2+, Cd2+, and Hg2+) in metalloproteins. Journal of inorganic biochemistry 71, 115-127 (1998).
37. J. A. Maier et al., ff14SB: improving the accuracy of protein side chain and backbone parameters from ff99SB. Journal of chemical theory and computation 11, 3696-3713 (2015).
38. R. L. Dunbrack Jr, F. E. Cohen, Bayesian statistical analysis of protein side-chain rotamer preferences. Protein Science 6, 1661-1681 (1997).
39. H. M. A. D. A. Case, K. Belfon, I. Y. Ben-Shalom, S. R. Brozell, D. S. Cerutti, T. E. Cheatham, III, G. A. Cisneros, V. W. D. Cruzeiro, T. A. Darden, R. E. Duke, G. Giambasu, M. K. Gilson, H. Gohlke, A. W. Goetz, R. Harris, S. Izadi, S. A. Izmailov, C. Jin, K. Kasavajhala, M. C. Kaymak, E. King, A. Kovalenko, T. Kurtzman, T. S. Lee, S. LeGrand, P. Li, C. Lin, J. Liu, T. Luchko, R. Luo, M. Machado, V. Man, M. Manathunga, K. M. Merz, Y. Miao, O. Mikhailovskii, G. Monard, H. Nguyen, K. A. O'Hearn, A. Onufriev, F. Pan, S. Pantano, R. Qi, A. Rahnamoun, D. R. Roe, A. Roitberg, C. Sagui, S. Schott-Verdugo, J. Shen, C. L. Simmerling, N. R. Skrynnikov, J. Smith, J. Swails, R. C. Walker, J. Wang, H. Wei, R. M. Wolf, X. Wu, Y. Xue, D. M. York, S. Zhao, and P. A. Kollman. (2021).
40. W. L. Jorgensen, Quantum and statistical mechanical studies of liquids. 10. Transferable intermolecular potential functions for water, alcohols, and ethers. Application to liquid water. Journal of the American Chemical Society 103, 335-340 (1981).
41. F. Neese, F. Wennmohs, U. Becker, C. Riplinger, The ORCA quantum chemistry program package. J Chem Phys 152, 224108 (2020).
42. A. D. Becke, Density-functional exchange-energy approximation with correct asymptotic behavior. Phys Rev A Gen Phys 38, 3098-3100 (1988).
43. F. Weigend, R. Ahlrichs, Balanced basis sets of split valence, triple zeta valence and quadruple zeta valence quality for H to Rn: Design and assessment of accuracy. Phys Chem Chem Phys 7, 3297-3305 (2005).
44. J. L. Whitten, Coulombic potential energy integrals and approximations. The Journal of Chemical Physics 58, 5 (1972).
45. B. I. Dunlap, J. Connolly, J. Sabin, On some approximations in applications of X a theory. The Journal of Chemical Physics 71, 3396-3402 (1979).
46. F. Neese, An improvement of the resolution of the identity approximation for the formation of the Coulomb matrix. J Comput Chem 24, 1740-1747 (2003).
47. D. A. Pantazis, X. Y. Chen, C. R. Landis, F. Neese, All-Electron Scalar Relativistic Basis Sets for Third-Row Transition Metal Atoms. J Chem Theory Comput 4, 908-919 (2008).
48. F. Weigend, Accurate Coulomb-fitting basis sets for H to Rn. Phys Chem Chem Phys 8, 1057-1065 (2006).
49. F. Neese, Prediction of molecular properties and molecular spectroscopy with density functional theory: From fundamental theory to exchange-coupling. Coordination Chemistry Reviews 253, 526-563 (2009).
50. E. v. Lenthe, E.-J. Baerends, J. G. Snijders, Relativistic regular two-component Hamiltonians. The Journal of chemical physics 99, 4597-4610 (1993).
51. E. van Lenthe, E.-J. Baerends, J. G. Snijders, Relativistic total energy using regular approximations. The Journal of chemical physics 101, 9783-9792 (1994).
52. C. van Wüllen, Molecular density functional calculations in the regular relativistic approximation: Method, application to coinage metal diatomics, hydrides, fluorides and chlorides, and comparison with first-order relativistic calculations. The Journal of chemical physics 109, 392-399 (1998).
53. S. Grimme, J. Antony, S. Ehrlich, H. Krieg, A consistent and accurate ab initio parametrization of density functional dispersion correction (DFT-D) for the 94 elements H-Pu. J

Chem Phys 132, 154104 (2010).
54. A. D. Becke, E. R. Johnson, A density-functional model of the dispersion interaction. J Chem Phys 123, 154101 (2005).
55. E. R. Johnson, A. D. Becke, A post-Hartree-Fock model of intermolecular interactions. J Chem Phys 123, 24101 (2005).
56. E. R. Johnson, A. D. Becke, A post-Hartree-Fock model of intermolecular interactions: inclusion of higher-order corrections. J Chem Phys 124, 174104 (2006).
57. V. Barone, M. Cossi, Quantum calculation of molecular energies and energy gradients in solution by a conductor solvent model. The Journal of Physical Chemistry A 102, 1995-2001 (1998).
58. M. Garcia-Ratés, F. Neese, Effect of the Solute Cavity on the Solvation Energy and its Derivatives within the Framework of the Gaussian Charge Scheme. Journal of Computational Chemistry 41, 922-939 (2020).
59. A. Schweiger, G. Jeschke, Principles of pulse electron paramagnetic resonance. (Oxford University Press on Demand, 2001).
60. S. A. Dikanov, Y. Tsvetkov, Electron spin echo envelope modulation (ESEEM) spectroscopy. (CRC press, 1992).
61. P. Höfer, A. Grupp, H. Nebenführ, M. Mehring, Hyperfine sublevel correlation (hyscore) spectroscopy: a 2D ESR investigation of the squaric acid radical. Chemical physics letters 132, 279-282 (1986).
62. E. Davies, A new pulse ENDOR technique. Physics Letters A 47, 1-2 (1974).
63. A. Tyryshkin, S. Dikanov, D. Goldfarb, Sum combination harmonics in four-pulse ESEEM spectra. Study of the ligand geometry in aqua-vanadyl complexes in polycrystalline and glass matrices. Journal of Magnetic Resonance, Series A 105, 271-283 (1993).
64. S. Stoll, in Methods in enzymology. (Elsevier, 2015), vol. 563, pp. 121-142.
65. J. L. Wimmer et al., Energy at Origins: Favorable Thermodynamics of Biosynthetic Reactions in the Last Universal Common Ancestor (LUCA). Frontiers in microbiology, 3903 (2021).
66. R. Conrad, W. Seiler, Methane and hydrogen in seawater (Atlantic Ocean). Deep Sea Research Part A. Oceanographic Research Papers 35, 1903-1917 (1988).
67. C. N. Pace, F. Vajdos, L. Fee, G. Grimsley, T. Gray, How to measure and predict the molar absorption coefficient of a protein. Protein science 4, 2411-2423 (1995).
68. K. C. Ryan et al., Nickel superoxide dismutase: structural and functional roles of His1 and its H-bonding network. Biochemistry 54, 1016-1027 (2015).
69. H.-I. Lee, J.-W. Lee, T.-C. Yang, S.-O. Kang, B. M. Hoffman, ENDOR and ESEEM investigation of the Ni-containing superoxide dismutase. JBIC Journal of Biological Inorganic Chemistry 15, 175-182 (2010).
70. S. Dikanov, Y. D. Tsvetkov, M. Bowman, A. Astashkin, Parameters of quadrupole coupling of 14N nuclei in chlorophyll a cations determined by the electron spin echo method.

Chemical Physics Letters 90, 149-153 (1982).
71. H. L. Flanagan, D. J. Singel, Analysis of 14N ESEEM patterns of randomly oriented solids. The Journal of chemical physics 87, 5606-5616 (1987).
72. S. Grimaldi et al., QH·-Ubisemiquinone Radical in the bo 3-Type Ubiquinol Oxidase Studied by Pulsed Electron Paramagnetic Resonance and Hyperfine Sublevel Correlation Spectroscopy. Biochemistry 40, 1037-1043 (2001).
73. D. T. Edmonds, Nuclear quadrupole double resonance. Physics Reports 29, 233-290 (1977).
74. K. Fukui, H. Ohya-Nishiguchi, H. Kamada, Electron spin echo envelope modulation study on oxovanadium (IV)-porphyrin complexes: reinvestigation of hyperfine and quadrupole couplings of pyrrole nitrogen. The Journal of Physical Chemistry 97, 11858-11860 (1993).
75. H.-I. Lee, K. S. Thrasher, D. R. Dean, W. E. Newton, B. M. Hoffman, 14N Electron Spin—Echo Envelope Modulation of the S=3/2 Spin System of the Azotobacter vinelandii Nitrogenase Iron—Molybdenum Cofactor. Biochemistry 37, 13370-13378 (1998).
76. L. L. Yap, R. I. Samoilova, R. B. Gennis, S. A. Dikanov, Characterization of the exchangeable protons in the immediate vicinity of the semiquinone radical at the QH site of the cytochrome bo3 from Escherichia coli. Journal of Biological Chemistry 281, 16879-16887 (2006).
77. S. A. Dikanov, A. M. Tyryshkin, I. Felli, E. J. Reijerse, J. Huttermann, C-band ESEEM of strongly coupled peptide nitrogens in reduced two-iron ferredoxin. Journal of Magnetic Resonance, Series B 108, 99-102 (1995).
78. S. A. Dikanov, M. K. Bowman, Cross-peak lineshape of two-dimensional ESEEM spectra in disordered S=12, I=12 spin systems. Journal of Magnetic Resonance, Series A 116, 125-128 (1995).
79. A. Tyryshkin, S. Dikanov, D. Goldfarb, Sum combination harmonics in four-pulse ESEEM spectra. Study of the ligand geometry in aqua-vanadyl complexes in polycrystalline and glass matrices. Journal of Magnetic Resonance, Series A 105, 271-283 (1993).
80. C. S. Burns et al., Molecular features of the copper binding sites in the octarepeat domain of the prion protein. Biochemistry 41, 3991-4001 (2002).
81. S. A. Dikanov, M. K. Bowman, Determination of ligand conformation in reduced [2Fe-2S] ferredoxin from cysteine β-proton hyperfine couplings. JBIC Journal of Biological Inorganic Chemistry 3, 18-29 (1998).
82. D. R. Kolling, R. I. Samoilova, A. A. Shubin, A. R. Crofts, S. A. Dikanov, Proton Environment of Reduced Rieske Iron—Sulfur Cluster Probed by Two-Dimensional ESEEM Spectroscopy. The Journal of Physical Chemistry A 113, 653-667 (2009).
83. J. D. Kim et al., Minimal heterochiral de novo designed 4Fe-4S binding peptide capable of robust electron transfer. Journal of the American Chemical Society 140, 11210-11213 (2018). https://pubs.acs.org/doi/pdf/10.1021/jacs.8b07553

While certain of the preferred embodiments of the present invention have been described and specifically exemplified above, it is not intended that the invention be limited to such embodiments. Various modifications may be made thereto without departing from the scope and spirit of the present invention, as set forth in the following claims.

### Embodiments

1. An isolated NB-2Ni peptide comprising at least 13 amino acids coordinating a di-nickel cluster which catalyzes production of H₂ in the presence of a suitable electron donor.

2. The peptide of embodiment 1 comprising at least one of SEQ ID NOS: 1 to 13 or a sequence 90% identical thereto which is synthetic and present in an aqueous solution.

3. An isolated polynucleotide encoding the polypeptide of embodiment 1 or embodiment 2.

4. A cell comprising the isolated polynucleotide of embodiment 3.

5. The cell of embodiment 4, being selected from the group consisting of a bacterial cell, a cyanobacterial cell, an algal cell, a fungal cell, and a plant cell.

6. A method of generating hydrogen, the method comprising combining the peptide of any of embodiments 1 or 2 with an electron donor so as to generate an electron transfer chain, wherein said electron transfer chain is configured such that said electron donor is capable of donating electrons to said peptide, thereby generating hydrogen.

7. The method of embodiment 6, wherein said electron donor is selected from the group consisting of a biomolecule, a chemical, water, an electrode and a combination of the above.

8. The method of embodiment 6, wherein hydrogen is generated using electrochemical reduction.

9. The method of embodiment 6, wherein hydrogen is generated using photochemical reduction.

10. The method of embodiment 6, wherein the generating hydrogen is effected under anaerobic conditions.

11. The method of any of embodiments 6-10, further comprising harvesting the hydrogen following the generating.

12. The method of embodiment 7, wherein said combining is effected in a cell-free system.

13. The method of embodiment 7, wherein said combining is effected in a cellular system.

14. The method of embodiment 13, wherein said cellular system is selected from the group consisting of a bacteria, an algae and a plant.

16. A system comprising the peptide of any one of embodiments 1 or 2 and an electron donor.

17. The system of embodiment 16, wherein said electron donor comprises an agent selected from the group consisting of a biomolecule, a chemical, water, an electrode and any combination of the above.

18. The system of embodiment 17, wherein said biomolecule is comprised in particles.

19. The system of embodiment 17, being expressed in cells.

20. A bioreactor for producing hydrogen, comprising: a vessel holding a hydrogen producing system, said system comprising a suspension of the peptide of any of embodiments 1 toy; a light providing apparatus comprising an optic fiber, said light providing apparatus being configured to provide light of a selected spectrum to said system; and a gas liquid separation membrane for separating gas leaving the suspension from said suspension.

21. The bioreactor of embodiment 20, wherein said system comprises a suspension of cells.

What is claimed is:

1. An isolated NB-2Ni peptide comprising at least 13 amino acids coordinating a di-nickel cluster which catalyzes production of H₂ in the presence of a suitable electron donor.

2. The peptide of claim 1 comprising at least one of SEQ ID NOS: 1 to 13 or a sequence 90% identical thereto which is synthetic and present in an aqueous solution.

3. An isolated polynucleotide encoding the polypeptide of claim 2.

4. A cell comprising the isolated polynucleotide of claim 3.

5. The cell of claim 4, being selected from the group consisting of a bacterial cell, a cyanobacterial cell, an algal cell, a fungal cell, and a plant cell.

6. The peptide of claim 2, wherein said peptide is fused to a light harvesting protein or chemical.

7. The peptide of claim 6, wherein the light harvesting protein or chemical is selected from PS1 and PS2, green fluorescent protein, orange fluorescent protein and derivatives, Eosin Y, Fluorescein, Ferredoxin.

8. The peptide of claim 2 wherein said peptide is fused to biotin.

9. The peptide of claim 8, wherein the fusion protein comprises SEQ ID NO: 14 or 15 or a sequence 90% identical thereto.

10. A method of generating hydrogen, the method comprising combining the peptide of claim 2 with an electron donor so as to generate an electron transfer chain, wherein said electron transfer chain is configured such that said electron donor is capable of donating electrons to said peptide, thereby generating hydrogen.

11. The method of claim 10, wherein said electron donor is selected from the group consisting of a biomolecule, a chemical, water, an electrode and a combination of the above.

12. The method of claim 10, wherein hydrogen is generated using electrochemical reduction.

13. The method of claim 10, wherein hydrogen is generated using photochemical reduction.

14. The method of claim 10, wherein the generating hydrogen is effected under anaerobic conditions.

15. The method of claim 10, further comprising harvesting the hydrogen following the generating.

16. The method of claim 11, wherein said combining is effected in a cell-free system.

17. The method of claim 11, wherein said combining is effected in a cellular system.

18. The method of claim 17, wherein said cellular system is selected from the group consisting of a bacteria, an algae and a plant.

19. A system comprising the peptide of claim 2 and an electron donor.

20. A bioreactor for producing hydrogen, comprising: a vessel holding a hydrogen producing system, said system comprising a suspension of the peptide of claim 2; a light providing apparatus comprising an optic fiber, said light providing apparatus being configured to provide light of a selected spectrum to said system; and a gas liquid separation membrane for separating gas leaving the suspension from said suspension.
